(19) United States
(12) Patent Application Publication
(10) Pub. No.: US 2024/0216311 A1
(43) Pub. Date: Jul. 4, 2024

(54) FULLY-DILUTABLE, SELF-MICROEMULSIFYING DELIVERY SYSTEMS (SMEDDS) FOR POORLY WATER-SOLUBLE POLAR SOLUTES

(71) Applicant: THE GOVERNING COUNCIL OF THE UNIVERSITY OF TORONTO, Toronto (CA)

(72) Inventors: Mehdi NOURAEI, Toronto (CA); Edgar ACOSTA, Toronto (CA); Yu-Ling CHENG, Toronto (CA); Levente DIOSADY, North York (CA); Venketeshwer RAO, Barrie (CA)

(73) Assignee: THE GOVERNING COUNCIL OF THE UNIVERSITY OF TORONTO, Toronto, ON (CA)

(21) Appl. No.: 18/269,712

(22) PCT Filed: Dec. 13, 2021
(86) PCT No.: PCT/CA2021/051794

§ 371 Date: Jun. 26, 2023

Related U.S. Application Data
(60) Provisional application No. 63/132,683, filed on Dec. 31, 2020.

(51) Int. Cl. A61K 31/192; A61K 9/00; A61K 9/107; A61K 9/48; A61K 31/00; A61K 31/05

(52) U.S. Cl. CPC A61K 31/192; A61K 9/0014; A61K 9/0053; A61K 9/1075; A61K 9/4875; A61K 31/05; A61K 31/658

(57) ABSTRACT

A fully dilutable in aqueous phase self-microemulsifying system for the delivery of one or more polar oil active compounds having a positive characteristic curvature (Cc), comprising: a lecithin compound; a hydrophilic linker or a combination two or more hydrophilic linkers, the hydrophilic linker or the combination of two or more hydrophilic linkers having one having one hydrocarbon group with 6 to 10 carbon atoms, and the hydrophilic linker or the combination of two or more HLs having a Cc of about −5 or more negative than about −5; and a carrier oil.

### FIELD OF THE INVENTION

The present invention relates to surfactant and oil solutions containing a dissolved pharmaceutical, food, cosmeceutical, biocide, or preservative compounds that are sparingly water-soluble and having polar oil characteristics. The disclosed solutions are designed to form microemulsions upon addition of an aqueous phase to deliver polar components to organisms or tissues, resulting in delivery systems for topical, transdermal, oral, buccal, vaginal, nasal, and ophthalmic applications, as well as food and agricultural applications.

### BACKGROUND OF THE INVENTION

Acosta and Nouraei [1] reviewed the state of the art on delivery systems, particularly for oral delivery applications in the food industry. The review points out that many delivery systems have not made it to the market because of their complexity in production, their low loading capacity, the use of expensive ingredients, and the use of non food-grade ingredients with unknown safety profile. Therefore, the need to use delivery systems that could be as concentrated as possible, using safe food-grade and preferably plant-derived ingredients, is advantageous towards finding a viable commercialization route. Among the possible delivery systems that could fit these advantageous characteristics, self-emulsifying and self-microemulsifying systems are of interest because their nanoscale size is often required to improve the uptake and bioavailability of the drug or active ingredient. Acosta and Nouraei defined microemulsions as Surfactant-Oil-Water (SOW) systems that exist in thermodynamic equilibrium with sizes often ranging between 1 and 100 nm. The authors further indicate that sizes lower than 500 nm are required for uptake by the intestines. Furthermore, the authors indicated that small drop sizes in delivery systems are always desired to improve the surface area/volume ratio of the delivery system (˜ 6/diameter of the delivery system), particularly for systems that may experience slow mass transfer. The authors further point to two manufacturing advantages of microemulsions over conventional emulsions. The first advantage is that self-microemulsifying and self-emulsifying systems do not need specialized high shear equipment (homogenizer, colloidal mills, and others) to produce the delivery systems, and simple mild mixing is enough to produce these delivery systems. The second advantage of self-microemulsifying systems is that microemulsions, existing in thermodynamic equilibrium, do not need coating agents to stabilize the diluted product, which is important to economically produce delivery systems with 1-50 nm scales.

Self-microemulsifying drug delivery systems (SMEDDS) are mixtures of surfactants (or surfactants+linkers) and oils that, upon dilution with an aqueous phase, form microemulsions with sizes often ranging in the 1-200 nm range. The smaller drop size (1-200 nm) of SMEDDS, compared to self-emulsifying drug delivery systems (SEDDS, 200 nm-1000 nm), gives a larger surface area to volume ratio for SMEDDS and enables the transport of microemulsion environments through tight pores. For transdermal delivery, most of the pore sizes available for drug delivery are smaller than 30 nm, and only soft delivery systems like soft vesicles or microemulsions can reach those pores, preferably compositions with sizes of 10 nm or smaller [2]. A similar pore size of 10 nm has been reported for intestinal tissue permeation [3]. The epithelial tissue of the bulbar conjunctiva can have pore sizes as large as 7.5 nm [4].

Given the mesh-like structure of the mucous layer surrounding the intestinal epithelial tissue, particles with up to 200 nm are preferentially retained in that zone of the intestine, allowing for larger release time [5]. The same principle of particle transport and retention in mucous layers applies to other wet epithelial tissue such as those lining the buccal cavity, the vaginal cavity, the lungs and airways, and the stomach [6]. SMEDDS are ideal delivery systems in this regard as they can achieve particle sizes of 200 nm or smaller. The high concentration of oil and surfactants in water-free preconcentrates of SMEDDS enables ease of manufacture and high loading capacity of drugs with low water solubility. The water-free environment of SMEDDS is also beneficial in preventing microbial growth, giving SMEDDS products greater biological stability.

The patent literature teaches of various examples of self-emulsifying systems of preconcentrates for drug delivery. The application WO/2018/011808 describes the use of PEG-based surfactants such as Cremophor EL and Polysorbate 80 to design preconcentrates for the delivery of cannabinoids that form emulsions with 10 nm-100 μm in size. The USPTO application 20190015346A1 discloses the use of preconcentrates that are also prepared with PEG-based surfactants such as Lauroyl polyoxylglycerides (PEG-32 esters) as SEDDS for cannabinoids. The U.S. Pat. No. 10,245,273 discloses the formulation of SMEDDS and SEDDS for the delivery of testosterone esters using a mixture of hydrophilic and lipophilic surfactants, where the hydrophilic surfactant is PEG-based, preferentially Cremophor RH 40 (PEG-40 Hydrogenated Castor Oil). U.S. Pat. No. 9,511,078 discloses the formulation of SEDDS with 50 nm to 800 nm in size for the delivery of poorly soluble drugs using propylene glycol (PPG) monocaprylate solvent and a PEG-based emulsifier. U.S. Pat. No. 9,918,965 discloses the formulation of SEDDS and SMEDDS for diindolylmethane and associated components via the combination of two emulsifiers, one lipophilic emulsifier with HLB lower than 7, including lecithin components, and one hydrophilic emulsifier with HLB higher than 7, where the preferred embodiments and examples make use of PEG-based hydrophilic emulsifiers. U.S. Pat. No. 8,790,723 discloses a self-nano emulsified drug delivery system (SNEDDS) produced with a mixture of a low HLB surfactant and a high HLB surfactant, Cremophor EL (PEG 35 ester of castor oil). U.S. Pat. No. 8,728,518 discloses SEDDS compositions for butylphthalide containing an emulsifier agent that may include lecithin but is preferably a PEG ester of castor oil or a PEG ester of glyceryl caprylate/caprate. U.S. Pat. No. 7,022,337 discloses self-emulsifying formulations for fenofibrate delivery and its derivatives using a combination of fenofibrate solubilizers (mainly PEG and polypropylene glycol or PPG compounds), stabilizers against crystallization (mainly alcohols and long-chain fatty acids), and surfactants, including lecithin among the possible candidates. U.S. Pat. No. 6,982,282 discloses self-emulsifying parenteral delivery systems for chemotherapeutics using, preferably. PEGylated surfactants. U.S. Pat. No. 7,419,996 discloses self-emulsifying systems for the delivery of benzimidazole using aprotic solvents combined with mixtures of sorbitan monooleate and PEG20-sorbitan monooleate. U.S. Pat. No. 6,960,563 discloses self-emulsifying cyclosporin delivery systems prepared with ethanol as a hydrophilic solvent and PEG-glycerol trioleate as an emulsifier. U.S. Pat. No. 8,962,696 discloses the formulation of self-microemulsifying delivery systems for propofol using PEG-containing surfactants. US patent application 20190216869A1 discloses the formulation of self-emulsifying delivery systems for cannabinoids using a mixture of cosolvents (including ethylene glycol, polyethylene glycol, alcohols, and PEGs), surfactants (with HLB less than 8 and between 9 and 20), and water. US patent application 20190111021A1 discloses self-emulsifying compositions to deliver tocotrienol using a carrier oil and a mixture of sorbitan monolaurate and PEG-20 sorbitan monooleate. US patent application 20190060300 discloses self-emulsifying compositions to deliver CB2 Receptor Modulators using a mixture of a surfactant with HLB<9) (including lecithin among the candidates) and a surfactant with HLB>13, citing preferred compositions of PEG-based surfactants with 15 ethylene glycol groups or more. US patent application 20180250262A1 discloses self-emulsifying compositions for the delivery of cannabinoids using a mixture of sesame oil, cyclodextrin, glyceryl behenate, lecithin, and PEG-6 caprylic/capric glycerides. US patent application 20190183838A1 discloses SEDDS, SNEDDS, and SMEDDS compositions for the delivery of polyunsaturated fatty acids and its esters using at least one surfactant of ionic, nonionic, or zwitterionic nature, including examples containing lecithin as a surfactant, PEG-based surfactants (Tween 20, Tween 80) and short-chain alcohols, polyethylene glycol (PEG) and propylene glycol (PPG) as cosolvents. US patent application 20180071210A1 discloses SEDDS compositions to deliver cannabinoids using PEG-PPG block copolymer surfactants and a polar solvent. US patent application 20140357708A1 discloses self-emulsifying compositions to deliver cannabinoids using triglycerides as a carrier oil to promote chylomicron/lipoprotein delivery (lymphatic transport) and reduce hepatic first-pass metabolism; and using lecithin, PEG-based surfactants, and C18+ polyglycerol surfactants to facilitate the self-emulsification process.

The numerous examples drawn from the patent literature reveal various important trends. First, that self-emulsification or self-microemulsion is an active area of development given its success in improving the uptake of poorly water-soluble compounds. Second, that the formulations tend to be comprised of zwitterionic surfactants such as lecithin or nonionic surfactants such as PEG-based surfactants and often rely on a combination of surfactants to achieve the desired performance. In several cases, these combinations are guided by the presence of a surfactant with HLB (hydrophilic-lipophilic balance) less than 10 and one with HLB greater than 10. Finally, most examples use PEG-based surfactants, particularly surfactants with 15 or more ethylene glycol units. One aspect missing in the patent literature cited above is any data or specifics about the dilutability of the disclosed compositions. The work of Chu et al. showed that the formation of self-emulsifying systems depends on the composition of the aqueous phase and the dilution ratio [7]. After searching the patent literature, only US patent application 20190008770 and U.S. Pat. No. 7,182,950 addressed the dilution process, claiming full dilutability. U.S. Pat. No. 7,182,950 uses ternary phase diagrams including a vertex of surfactant composition, oil composition, and aqueous phase composition to illustrate the complexity of producing fully dilutable delivery systems and that only certain compositions of surfactants and oil can be diluted with specific compositions of an aqueous phase and hydrophilic cosolvents that include ethanol and glycerol. However, in physiological conditions, the aqueous phases diluting the composition do not contain these solvents, and instead, they are aqueous solutions containing salts, lipids, and proteins. US patent application 20190008770 does not introduce ternary phase diagrams to explain the dilution process but mentions dilution with water and hydrophilic cosolvents ethanol, glycerol, propylene glycol, and PEG to achieve full dilutability.

The use of PEG-based surfactants, especially those with more than 10 ethylene glycol groups, is often justified by the low toxicity of those surfactants and because they impart stealth characteristics to the delivery system [8]. This stealth characteristic means that delivery systems with PEG-based surfactants tend to bypass metabolic pathways, leading to extended circulation time in the blood. For oral delivery applications, however, being stealth is not desirable because it interferes with chylomicron assembly, which enables lymphatic transport. For example, Pluronic L-81, a PPG-PEG block copolymer, inhibits the uptake of beta-carotene when compared to surfactant-free delivery, while a simulated bile salt delivery system enhances the bioavailability of beta-carotene [9]. The stealth nature of PPG and PEG components is partially due to the lack of enzymes that can hydrolyze these components, considering that they are not found in nature. Nevertheless, due to the multiple and repeated exposure to PEG components, there is increasing evidence that humans are adapting to these components, and there are more frequent reports of PEG-induced Accelerated Blood Clearance (ABC) and an autoimmune response called the complement (C) activation-related pseudoallergy (CARPA) [10]. One the biggest draw back of the Pfizer-BioNTech COVID-19 vaccines is that they may cause allergic reactions due to presence of the PEG in its formulation. Because of these reasons, self-emulsifying and self-microemulsifying compositions that are free of PEG and PPG compounds are considered advantageous as delivery systems.

Acosta and Yuan (U.S. Pat. No. 9,918,934) disclosed microemulsion-based delivery compositions containing a lecithin compound as the main surfactant, a lipophilic linker having C12+alkyl chain with HLB 5 or less; and C6-C9 surfactant-like hydrophilic linker. The disclosed formulations are PEG-free, PPG-free, and free of short-chain alcohol and medium-chain alcohol. The disclosures in this patent, however, do not include SMEDDS nor any description on how to produce water-free formulations that are fully dilutable.

For selected delivery applications, such as subcutaneous, buccal, topical, ophthalmic, and vaginal delivery is desirable for the delivery system to have solid-like (gel) and extended-release properties. These desirable properties allow for a concentrated dose to be safely placed next to an epithelial tissue for extended-release of safe and effective doses of a wide range of actives, including insulin and antimicrobials [11-14]. There are numerous gels systems designed to deliver actives; however, lecithin-based gels offer the advantage of facilitating food-grade formulations and enabling lymphatic transport. Current lecithin-based gels often use gelating agents to trap oils, emulsions, microemulsions, or even aqueous solutions, but not lecithin-based SMEDDS [15]. The advantage of incorporating SMEDDS into slow-release lecithin-based gel systems is that high concentrations of the drug can be loaded into the gel, allowing slow and continuous release without reaching potentially toxic high “dump” doses. Gelled SMEDDS have been reported as a solid-like alternative to liquid SMEDDS, and they are produced by embedding gel-forming polymers with the SMEDDS composition [16]. There are reports on the use of low molecular weight gelators such as 12-hydroxystearic acid (12-HSA) and beta-sitosterol to produce organogels of oil mixtures containing drugs to provide long release times[17,18]. However, the same references report that incorporating surfactants such as lecithin and polyglycerol esters reduce the mechanical strength of the gel and are, therefore, undesirable contaminants. These observations suggest that it is impossible to formulate a gelled SMEDDS with a low molecular weight gelator such as 12-HSA or phytosterols with extended-release properties. Perhaps due to this understanding in the field, no patents for gelled SMEDDS with low molecular weight gelators were found. The closest document is patent application WO2008037697A1 on novel organogel particles that describes the use of 12-HSA hot-diluted in oils and then incorporated into an aqueous solution containing surfactants under high intensity mixing to then produce gellosomes (dispersed gelled phases). However, the reported invention required the use of water to induce the formation of gellosomes, which is contrary to the idea of producing water-free gelled SMEDDS.

For food and pharmaceutical applications, encapsulation of the delivery systems is often necessary to protect the stomach lining from the active ingredients and to protect active ingredients from the acidic environment of the stomach. Therefore, encapsulated SMEDDS formulations are expected to produce useful formulas for various products, including nonsteroidal anti-inflammatory drugs (NSAIDs) that are known to affect the inner lining of the stomach. Initial attempts to encapsulate SMEDDS concentrated on filling gelatin capsules with SMEDDS. However, more recent attempts include the embedding SMEDDS into polymer matrices that provide temporal protection against release in the stomach [16,19]. One report using spray-drying to encapsulate SMEDDS employed dextrose as a coating agent, but this agent does not produce protection against acidic release (i.e., it is not an enteric coating)[20]. One desirable feature of spray-dried products is that it produces free-flowing powders that can be easily incorporated into food products, gel products, and pellets. Patent application US2018/0021349A1 discloses compositions of SMEDDS formulated with PEG-based surfactants mentioning potential encapsulation technologies, including spray-drying. However, the invention does not disclose enteric encapsulation compositions. Enteric encapsulation of microemulsions has been claimed in U.S. Pat. No. 6,280,770B1 accomplished by absorption of SMEDDS into a porous material with enteric protection properties. This brief review shows a clear gap in the technology for powder spray-dried SMEDDS formulations with enteric coating agents.

### Hydrophilic-Lipophilic Difference (HLD)) and Characteristic Curvature ((c)

Nouraei and Acosta produced the first example of lecithin+linkers fully dilutable formulation, which was designed via the hydrophilic-lipophilic difference (HLD) framework, requiring the measurement of the characteristic curvature (Cc) of the linkers and lecithin, and the equivalent alkane carbon number (EACN) of the oil. The authors indicated that the minimum lipophilic linker to lecithin ratio necessary to prevent highly viscous liquid crystals and gels was 1 part (by mass) of the lipophilic linker (sorbitan monooleate or glycerol monooleate) for 1 part of lecithin. Furthermore, the authors used the net-average curvature (NAC) model, associated with the HLD, to predict the 2-phase region of the ternary phase diagram. The authors used the HLD-NAC framework to identify a region of the ternary phase diagram with a fully-dilutable region suitable for SMEDDS formulations. The fully-dilutable composition disclosed by Nouraei and Acosta was comprised of lecithin as the main surfactant, glycerol monooleate as a lipophilic linker and polyglycerol caprylate (Dermofeel® G6CY) as a hydrophilic linker. The Cc of polyglycerol caprylate Dermofeel® G6CY is around −3. The composition was PEG-free, PPG-free, and free of medium and short-chain alcohols. Nouraei and Acosta highlighted the complex nature of the formulation, indicating that a change in the ratios among the linkers and lecithin was enough to eliminate the fully-dilutable path. The authors determined that the HLD value of the formulation can serve as a guideline to reach the conditions for full dilutability. The HLD is an empirical equation that relates the formulation conditions to the proximity to the surfactant phase inversion point, where HLD=0 [21]. For systems containing nonionic surfactants such as those employed in the lecithin-linker compositions:

$$HLD = b \cdot S - k \cdot EACN + Cc + {c}_{T} \cdot ( T - {25}^{∘} C . ) \qquad ( 1 )$$

where b, k and Cr are constants that depend on the surfactant used and the electrolyte dissolved in the aqueous phase. S is the salinity of the aqueous phase, normally expressed in g NaCl/100 mL for saline solutions. T is the temperature of the systems in Celsius. The Cc is the characteristic curvature of the surfactant, with more hydrophilic surfactants having more negative Cc values. For linear alkanes, EACN is simply the number of carbons in their chain, and for other oils, this value is determined experimentally using methods reported in the literature [22].

An advantageous feature of SMEDDS is delivering concentrated doses of actives through living tissues (animals, plants, and microbial species). This feature makes SMEDDS a desirable technology to incorporate pharmaceutical active ingredients, nutraceuticals, cosmeceuticals, and a wide range of biocides into pharmaceutical, food, cosmetic, cleaning and disinfecting, and agrochemical compositions. Many components of interest in medical, cosmetic, food and agricultural applications are not simple hydrocarbons with a defined EACN. Instead, many of these components are polar oils.

Polar oils are a broad class of oils consisting of a heteroatom-linked polar group attached to a nonpolar hydrocarbon, producing non-zero dipole moments and a non-zero polar surface area. Polar groups include carboxylic acids, alcohols, amines, amides, ethers, esters, aldehydes, and haloalkanes. The polarity of these oils allows them to segregate towards the oil-water interface, displaying a surfactant-like behavior and at the same time partition into the bulk oil phase, displaying an oil-like behavior. Polar oils have been found to have a positive value of Cc or a value of apparent EACN that is negative [23]. Formulating microemulsion systems (including SMEDDS) with polar oils remains a complex task, even for those skilled in the art [24].

FIG. 1 Illustrates the challenge of incorporating polar oils, in this case, ibuprofen (containing a carboxylic acid polar group), into the SMEDDS composition disclosed by Nouraei and Acosta [21]. The 10-10-80 (lecithin-glycerol monooleate-polyglycerol-6-caprylate) system of FIG. 1, formulated with a 75/25 surfactant mixture/oil (ethyl caprate) ratio (also known as a D75 SMEDDS), was fully dilutable in the absence of ibuprofen (top set of dilution vials). However, adding 5% ibuprofen to the SMEDDS disrupted the fully dilutable path (onset of phase separation) of the original SMEDDS. The introduction of a polar oil can induce a phase inversion of the surfactant into the oil. In HLD terms, this would represent a positive HLD shift. The 10-10-80 system, with HLD=−1.85 [21], was selected to mitigate this effect: however, not even this precaution prevented phase separation. Other attempts to restore the full dilutability included replacing ethyl caprate with mineral oil with high EACN (a negative HLD shift) and reducing the lecithin and lipophilic linker content to a minimum, all without success. The compositions disclosed herein represent unexpected solutions to this formulation challenge. The compositions disclosed herein overcome the challenge of formulating fully dilutable SMEDDS containing polar oil solutes.

### SUMMARY OF THE INVENTION

The present disclosure relates to lecithin-based, fully-dilutable self-microemulsifying drug delivery systems (SMEDDS) compositions used to solubilize and deliver poorly water-soluble polar active ingredients. The delivery can be via topical, transdermal, oral, transnasal, buccal, vaginal, subcutaneous, parenteral, and ophthalmic routes in humans and animals for food, cosmetic and pharmaceutical applications. The compositions described in this disclosure are also useful in delivering actives to plants, insects and microorganisms for agricultural, pest, and disease control. In embodiments, the lecithin-based SMEDDS compositions of the present disclosure are comprised of lecithin as the main surfactant, a hydrophilic linker (HL) comprising a C6-C10 surfactant with characteristic curvature (Cc) of about −5 or more negative than about −5 (also referred to as “extreme hydrophilic linker”), and a carrier oil phase. In aspects, the carrier oil phase has a positive equivalent alkane carbon number (EACN) such as alkyl esters of fatty acids, terpenes, essential oils and food-grade or pharma-grade hydrocarbons, or mixtures thereof that may be required to dissolve the polar oil solute in the SMEDDS. In aspects, the SMEDDS may also be comprised of a C10+ lipophilic linker having a characteristic curvature (Cc) more positive than +3. The disclosed fully-dilutable SMEDDS contains a poorly water-soluble polar oil as an active ingredient having water solubility lower than 1 wt %, log P greater than 1.5, and a positive characteristic curvature (Cc), or negative apparent EACN. The water-free SMEDDS are fully dilutable in isotonic solutions containing lipids and proteins typically found in biological fluids (i.e., intestinal fluids, CFS, tear fluid, saliva, sweat, plasma, blood and so forth), producing drop sizes of 1 to 200 nm. The SMEDDS are free of short (C1 to C3) chain alcohol, medium (C4 to C8) chain alcohol, PEG, PPG, PEG-based surfactants, and PPG-based surfactants.

The disclosed PEG-free and fully-dilutable lecithin-based SMEDDS increased the transdermal permeation of solutes that are sparingly soluble in water and that have polar oil characteristics. In another embodiment, the lecithin-based SMEDDS was shown to increase the absorption of the polar active ingredient via oral delivery, also producing a fast-acting transport of the polar active, whose plasma concentration remains relatively high for an extended period.

In another embodiment, the disclosed fully-dilutable lecithin-based SMEDDS further comprises a low molecular weight organic gelator to produce gelled SMEDDS that offer an extended-release of the active for over one day of release. These gelled SMEDDS compositions are useful to avoid potentially undesirable burst release effects and reduce frequent dosing of active compounds.

In another embodiment, the disclosed fully-dilutable lecithin-based SMEDDS further comprises a coating agent that imparts enteric protection during gastric passage. The composition is first diluted in an aqueous environment to generate a microemulsion containing a dispersion of the coating agent. The dispersion is then spray-dried to generate free-flowing encapsulated SMEDDS particles. These encapsulated SMEDDS particles are useful to incorporate SMEDDS into solid and semisolid products and tablets. The encapsulated SMEDDS protects the active from the gastric acid environment and protects the stomach lining from potential adverse effects induced by the delivered active.

Disclosed is a fully dilutable in aqueous phase self-microemulsifying system for the delivery of one or more polar oil active compounds having a positive characteristic curvature (Cc), comprising: (a) a lecithin compound; (b) a hydrophilic linker (HL) or a combination of two or more hydrophilic linkers (HLs), the HL or each of the HLs within the combination having one hydrocarbon group with at least 50% or more alkyl chain distribution between 6 to 10 carbon atoms (i.e., C6, C7, C8, C9 or C10), and the HL or the combination of two or more HLs having a Cc of about −5 or more negative than −5; and (c) a carrier oil.

In one aspect of the disclosed fully dilutable in aqueous phase self-microemulsifying system, the delivery is topical, transdermal, oral, transnasal, buccal, vaginal, subcutaneous, parenteral, ophthalmic, transepidermal, transmembrane, and/or intravenous.

In another aspect of the disclosed fully-dilutable in aqueous phase self-microemulsifying system, the lecithin compound concentration is about 1.5% to about 45% w/w.

In another aspect of the disclosed fully-dilutable in aqueous phase self-microemulsifying system, the lecithin compound is vegetable lecithin, animal lecithin or synthetic lecithin containing at least 50% w/w of a mixture of phosphatidylcholine, phosphatidylinositol, phosphatidylethanolamine, phosphatidylserine, and phosphatidic acid, and lysotecithins.

In another aspect of the disclosed fully-dilutable in aqueous phase self-microemulsifying system, the hydrophilic linker or the combination of two or more HLs is about 10 wt % to about 86 wt % of the system.

In another aspect of the fully dilutable in aqueous phase self-microemulsifying system, the combination of two or more HLs includes least one amphiphilic compound with a Cc less negative than about −5 and the Cc of the combination is about −5 or more negative than about −5.

In another aspect of the disclosed fully-dilutable in aqueous phase self-microemulsifying system, the hydrophilic linker or the combination of two or more HLs comprises one or more of C6-C10 alkyl polyphosphates, polyphosphonates, polycarboxylates, sulfosuccinates: glutamates, C6-C10 esters of polyhydric alcohols, polyvinyl alcohol, polyglycerols and their co-polymers with a degree of polymerization (n) higher than 2 (n>2), sucrose, maltose, oligosaccharides, polyglucosides (n>2), polyglucosamines, sorbitol, sorbitan, poly alpha hydroxy acids and their salts, C6-C10 amines, quaternary ammonium salts, amine oxides, C6-C10 alkyl aminopropionic acids, betaines, sulfobetaines, phosphatidylcholines, phosphatidyl glycerols, or mixtures thereof.

In another aspect of the disclosed fully-dilutable in aqueous phase self-microemulsifying system, the hydrophilic linker or at least one of the two or more HLs in the combination is a C6-C10 polyglycerol with a degree of polymerization n>2.

In another aspect of the disclosed fully-dilutable in aqueous phase self-microemulsifying system, the hydrophilic linker or at least one of the two or more HLs in the combination is disodium C6-C10 glutamate, polyglycerol-6-caprylate or polyglycerol-10 caprylate.

In another aspect of the disclosed fully-dilutable in aqueous phase self-microemulsifying system, the carrier oil has a positive equivalent alkane carbon number (EACN).

In another aspect of the disclosed fully-dilutable in aqueous phase self-microemulsifying system, the carrier oil concentration is about 10 wt % to about 70 wt %.

In another aspect of the disclosed fully-dilutable in aqueous phase self-microemulsifying system, the carrier oil comprises of alkyl esters of fatty acids, monoglycerides, diglycerides, triglycerides, alkanes, terpenes, or mixtures thereof.

In another aspect of the disclosed fully-dilutable in aqueous phase self-microemulsifying system, the self-microemulsifying system further includes the one or more polar oil active compounds having a positive characteristic curvature (Cc).

In another aspect of the disclosed fully-dilutable in aqueous phase self-microemulsifying system, the concentration of the one or more polar oil active compounds is about 0.01 wt % to about 80 wt %.

In another aspect of the disclosed fully-dilutable in aqueous phase self-microemulsifying system, each of the one or more polar oil active compounds having a positive characteristic curvature (Cc) has a log P greater than 1, molecular weight between 50 and 100,000 Daltons, a polar area greater than 0.0 Λ², an aqueous solubility less than about 1 wt %.

In another aspect of the disclosed fully-dilutable in aqueous phase self-microemulsifying system, the one or more polar oil active compounds having a positive characteristic curvature (Cc) includes one or more hydrogen bonding donor compounds selected from a group consisting of C5+ alcohols, amines, peptides, organic acids, anthranilic acids, aryl propionic acids, enolic acids, heteroaryl acetic acids, indole and indene acetic acids, salicylic acid derivatives, nucleic acids, alkylphenols, para-aminophenol derivatives, terpene phenolics, cannabinoids, alkaloids, peptides, and halogenated compounds.

In another aspect of the disclosed fully-dilutable in aqueous phase self-microemulsifying system, the one or more polar active compounds include ibuprofen, nonylphenol, cannabidiol, and eugenol.

In another aspect of the disclosed fully-dilutable in aqueous phase self-microemulsifying system, the aqueous phase is water, biological fluids, aqueous electrolyte solutions, carbonated drinks, fruit juices, or alcoholic beverages.

In another aspect of the disclosed fully-dilutable in aqueous phase self-microemulsifying system, the system further comprises a lipophilic linker.

In another aspect of the disclosed fully-dilutable in aqueous phase self-microemulsifying system, the lipophilic linker concentration is about 0.1 wt % to about 30.0 wt %.

In another aspect of the disclosed fully-dilutable in aqueous phase self-microemulsifying system, the lipophilic linker includes one or more ingredients selected from a group consisting of C12+ alcohols, fatty acids, monoglyceride, sorbitan ester, sucrose ester, glucose ester.

In another aspect of the disclosed fully-dilutable in aqueous phase self-microemulsifying system, the lipophilic linker includes one or more ingredients selected from a group consisting of dodecyl alcohol, oleyl alcohol, cholesterol, lauric acid, palmitic acid, oleic acid, omega 6-fatty acids, omega 3-fatty acids, esters of these fatty acids with sorbitol, maltitol, xylitol, isomalt, lactitol, erythritol, pentaerythritol, glycerol: for example, sorbitan monooleate, and glycerol monooleate.

In another aspect of the disclosed fully-dilutable in aqueous phase self-microemulsifying system, the system further comprises of a low molecular weight organogelator that imparts semisolid properties and produces a slow releasing profile of the one or more polar oil active compounds.

In another aspect of the disclosed fully-dilutable in aqueous phase self-microemulsifying system, the concentration of the organogelator is about 0.1 wt % to about 40.0 wt %.

In another aspect of the disclosed fully-dilutable in aqueous phase self-microemulsifying system, the organogelator includes one or more ingredients selected from sterol-based gelling agents, long-chain fatty acids, long-chain amines, and esters of long-chain fatty acids.

In another aspect of the disclosed fully-dilutable in aqueous phase self-microemulsifying system, the system further comprises an encapsulating agent that imparts solid-like properties and produce flowable powders that can form micellar solutions when diluted in aqueous environments.

In another aspect of the disclosed fully-dilutable in aqueous phase self-microemulsifying system, the concentration of the encapsulating agent is about 10% to about 90.0% wt.

In another aspect of the disclosed fully-dilutable in aqueous phase self-microemulsifying system, the encapsulating agent includes one or more ingredients selected from amphiphilic polymers with a glass transition temperature ranging from about 45° C. to about 99° C.

In another aspect of the disclosed fully-dilutable in aqueous phase self-microemulsifying system, the system comprises between 30 parts of a mixture of the lecithin and hydrophilic linker and 70 parts of the carrier oil (D30) and 90 parts of the mixture of lecithin and hydrophilic linker and 10 parts of the carrier oil (D90).

In another aspect of the disclosed fully-dilutable in aqueous phase self-microemulsifying system, the system comprises between 40 parts of a mixture of the lecithin and hydrophilic linker and 60 parts of the carrier oil (D40) and 80 parts of the mixture of lecithin and hydrophilic linker and 20 parts of the carrier oil (D80).

In another aspect of the disclosed fully-dilutable in aqueous phase self-microemulsifying system, the system is waterless.

In another aspect of the disclosed fully-dilutable in aqueous phase self-microemulsifying system, the system is free of polyethylene glycol, propylene glycol, and short and medium-chain alcohols.

In another aspect of the disclosed fully-dilutable in aqueous phase self-microemusifying system, the system has particle diameters smaller than 200 nm.

Disclosed is also a capsule comprising any one of the fully dilutable in aqueous phase self-microemulsifying systems of the present disclosure.

Disclosed herein is also a method of delivering one or more polar oil active compounds having a positive characteristic curvature (Cc) across an epithelium, the method comprising contacting the epithelium with a composition comprising the fully dilutable in aqueous phase, self-microemulsifying system according to the present disclosure. In aspects of the method, the composition is a cosmetic composition, a nutraceutical composition, a food composition or a pharmaceutical composition.

Disclosed is also a method of delivering one or more polar oil active compounds having a positive characteristic curvature (Cc) to a subject comprising administering to a subject a fully dilutable in aqueous phase self-microemulsifying system comprising: (a) a lecithin compound; (b) a hydrophilic linker (HL) or a combination of two or more hydrophilic linkers (HLs), the HL or each of the HLs within the combination having one hydrocarbon group with at least 50% or more alkyl chain distribution between 6 to 10 carbon atoms and the HL or the combination of two or more HLs having a Cc of about −5 or more negative than −5; (c) a carrier oil; and (d) the one or more polar oil active compounds having the positive Cc. In one aspect of this method the system is formulated for topical, transdermal, oral, buccal, vaginal, nasal, subcutaneous, parenteral, transepidermal, transmembrane and/or ophthalmic delivery. In another aspect, the fully dilutable in aqueous phase, self-microemulsifying system is any one of the systems of the present disclosure.

### BRIEF DESCRIPTION OF THE DRAWINGS

A detailed description of the preferred embodiments is provided herein below by way of example only and with reference to the following drawings, in which:

FIG. 1A shows the dilution of the 10-10-80 formulation at 75:25 Surfactant: Oil.

FIG. 1B shows the dilution of 10-10-80 formulation when loaded with 5% ibuprofen. SIF % represents the mass percentage of fed-state simulated intestinal fluid (SIF) in the diluted SMEDDS.

FIG. 2A shows the solubilization parameter for oil (heptane, diamonds) and water (squares) in middle phase microemulsions as a function of the salinity (g NaCl/100 mL) in the aqueous phase. The point of equal solubilization for oil and water is indicated as S*, the optimal salinity. The system corresponds to a 20 wt % Caprol® 6GC8 in mixture with the reference surfactant C9E5.

FIG. 2B shows the optimal salinity (S*) for microemulsions produced with heptane as the oil phase and mixtures of Caprol® 6GC8 and C9E5, as a function of the molar fraction of Caprol® 6GC8 in mixtures with C9E5.

FIG. 3A shows the optimal salinity (S*) for microemulsions produced with heptane as the oil phase and mixtures of ibuprofen and C9E5, as a function of the molar fraction of ibuprofen in mixtures with 5 wt % C9E5 in the aqueous phase.

FIG. 3B shows the optimal salinity (S*) for microemulsions produced with heptane as the oil phase and mixtures of nonylphenol and C9E5, as a function of the molar fraction of nonylphenol in mixtures with 5 wt % C9E5 in the aqueous phase.

FIG. 3C shows the optimal salinity (S*) for microemulsions produced with heptane as the oil phase and mixtures of eugenol and C9E5, as a function of the molar fraction of eugenol in mixtures with 5 wt % C9E5 in the aqueous phase.

FIG. 3D shows the optimal salinity (S*) for microemulsions produced with heptane as the oil phase and mixtures of benzocaine and C9E5, as a function of the molar fraction of benzocaine in mixtures with 15 wt % C9E5 in the aqueous phase.

FIG. 3E shows the optimal salinity (S*) for microemulsions produced with cyclohexane as the oil phase and mixtures of cannabidiol (CBD) and C9E5, as a function of the molar fraction of CBD in mixtures with 7 wt % C9E5 in the aqueous phase.

FIG. 4 shows the ternary phase diagram for a SMEDDS system formulated with soy bean lecithin (10 parts), lipophilic linker (10 parts), and conventional hydrophilic linker Dermofeel® G6CY (Cc=−3) (80 parts), and containing 5% ibuprofen, and ethyl caprate as carrier (solvent) oil.

FIG. 5 shows the ternary phase diagram for a fully dilutable SMEDDS system formulated with soy bean lecithin (10 parts), and extreme hydrophilic linker Polyaldo® 10-1-CC (Cc=−7.4) (90 parts) and containing 5% ibuprofen and ethyl caprate as carrier (solvent) oil.

FIG. 6 shows the ternary phase diagram for a fully dilutable SMEDDS system formulated with soybean lecithin (15 parts), lipophilic linker Peceol™ (15 parts), and extreme hydrophilic linker Caprol® 6GC8 (Cc=−6.4) (70 parts), and containing 5% ibuprofen, and ethyl caprate as carrier (solvent) oil.

FIG. 7 shows the ternary phase diagram for a fully dilutable SMEDDS system formulated with soy bean lecithin (10 parts), and extreme hydrophilic linker Polyaldo®10-1-CC (Cc=−7.4) (90 parts), and containing 5% cannabidiol (CBD), and Limonene as carrier (solvent) oil.

FIG. 8. Top picture: red channel image of the water dilution of D70 Lecithin-Polyaldo®10-1-CC-limonene formulation containing 5% CBD. Bottom picture: blue channel image of the water dilution of D70 Lecithin-Polyaldo®10-1-CC-limonene formulation containing 5% CBD.

FIG. 9 shows the cumulative transdermal permeation of nonylphenol (NP) through excised pig skin. Circles correspond to 10% NP formulated in a SMEDDS(i) produced 10 parts lecithin+90 parts Polyaldo®10-1-CC, and ethyl caprate following a D50 dilution line and diluted with 70 parts FeSSIF and 30 parts of SMEDDS(i). Squares correspond to 10% NP formulated in a SMEDDS(ii) produced 15 parts lecithin+15 parts Peceol™+70 parts Polyaldo®10-1-CC, and ethyl caprate following a D50 dilution line and diluted with 70 parts FeSSIF and 30 parts of SMEDDS(ii). Triangles correspond to 10% NP diluted in a carrier oil (ethyl caprate) only.

FIG. 10 shows the plasma concentration of ibuprofen in male Sprague-Dawley rats after an oral dose of 25 mg/kg ibuprofen. Circles correspond to the ibuprofen formulated in the SMEDDS composition of Example 5. Triangles correspond to ibuprofen formulated as a suspension (control or reference case) in 0.1% (w/v) of sodium carboxymethyl cellulose solution. The dashed line corresponds to the first order and single compartment pharmacokinetic model fit of the SMEDDS plasma concentration data. The solid line represents the first order and single compartment pharmacokinetic model fit of the plasma concentration data obtained with the control case.

FIG. 11 shows the elastic (G′) and shear (G″) moduli obtained during the heating cycle experiments for a gelled SMEDDS prepared with equal parts of Lecithin-HL mixture and ethyl caprate and containing 5 wt % nonylphenol and 10 wt % HSA gelator. The Lecithin-HL mixture contained (10 parts) lecithin and extreme hydrophilic linker Polyaldo® 10-1-CC (90 parts). Rheological measurements were conducted using a heating rate of 0.8° C./min, 10 rad/s, and 0.1% strain.

FIG. 12 shows the release of nonylphenol into FeSSIF as a function of the square root of release time from a gelled SMEDDS prepared with equal parts of Lecithin-HL mixture and ethyl caprate and containing 5 wt % nonylphenol and 10 wt % HSA gelator. The Lecithin-HL mixture contained (10 parts) lecithin and extreme hydrophilic linker Polyaldo® 10-1-CC (Cc=−7.4) (90 parts).

FIG. 13 shows the elastic (G′) and shear (G″) moduli obtained during the heating cycle experiments for a gelled SMEDDS prepared with equal parts of Lecithin-HL mixture and ethyl caprate and containing 5 wt % nonylphenol and 18 wt % (squares) and 20 wt % (circles) of a 1:1 weight ratio mixture of β-sitosterol+γ-oryzanol used as the gelator mixture. The Lecithin-HL mixture contained (10 parts) lecithin and extreme hydrophilic linker Polyaldo®10-1-CC (90 parts). Rheological measurements were conducted using a heating rate of 0.8° C./min, 10 rad/s, and 0.1% strain.

FIG. 14 shows the release of nonylphenol into FeSSIF as a function of the square root of release time from a gelled SMEDDS prepared with equal parts of Lecithin-HL mixture and ethyl caprate, and containing 5 wt % nonylphenol and 18 wt % (squares) and 20 wt % (circles) of a 1:1 weight ratio mixture of β-sitosterol+γ-oryzanol used as the gelator mixture. The Lecithin-HL mixture contained (10 parts) lecithin and extreme hydrophilic linker Polyaldo®10-1-CC (90 parts).

FIG. 15.A shows the particle size distribution and angle of repose for the encapsulated D60 SMEDDS prepared with 10 parts (by mass) of lecithin and 90 parts Polyaldo® 10-1-CC (Cc=−7.4) using limonene as a carrier oil and containing 5% nonylphenol. Encapsulation was obtained via spray drying of 60 parts (by mass) of EUDRAGUARD®) (natural non-enteric coating agent) and 40 parts of the D60 SMEDDS.

FIG. 15.B shows the particle size distribution and angle of repose for the encapsulated D60 SMEDDS prepared with 10 parts (by mass) of lecithin and 90 parts Polyaldo®10-1-CC (Cc=−7.4) using limonene as a carrier oil and containing 5% nonylphenol. Encapsulation was obtained via spray drying of 60 parts (by mass) of EUDRAGIT® FL 30 D-55 (enteric coating agent) and 40 parts of the D60 SMEDDS.

FIG. 15C shows the particle size distribution and angle of repose for the encapsulated D60 SMEDDS prepared with 10 parts (by mass) of lecithin and 90 parts Polyaldo®10-1-CC (Cc=−7.4) using limonene as a carrier oil and containing 5% nonylphenol. Encapsulation was obtained via spray drying of 60 parts (by mass) of PROTECT™ ENTERIC (enteric coating agent) and 40 parts of the D60 SMEDDS.

FIG. 16 shows the plasma concentration of CBD in male Sprague-Dawley rats after an oral dose of 10 mg/kg CBD. Circles correspond to the CBD formulated in the 20% CBD-D70 SMEDDS composition of Example 16. Triangles correspond to the control case of CBD formulated as a 9.6 mg/ml solution in medium chain triglycerides (MCT). The squares correspond to the CBD formulated in encapsulated (powder) 20% CBD-D70 SMEDDS composition of Example 16.

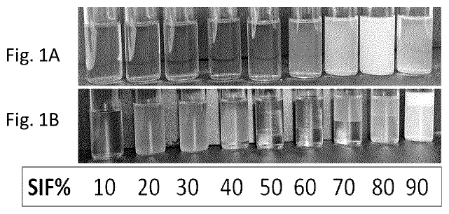

In the drawings, one embodiment of the invention is illustrated by way of example. It is to be expressly understood that the description and drawings are only for the purpose of illustration and as an aid to understanding and are not intended as a definition of the limits of the invention.

### DETAILED DESCRIPTION OF THE INVENTION

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods, devices and materials are now described. All technical and patent publications cited herein are incorporated herein by reference in their entirety. Nothing herein is to be construed as an admission that the disclosure is not entitled to antedate such disclosure by virtue of prior disclosure.

All numerical designations, e.g., Characteristic curvature (Cc), pH, temperature, time, concentration and molecular weight, including ranges, are approximations which are varied (+) or (−) by increments of 1.0 or 0.1, as appropriate, or alternatively by a variation of +/−20%, +/−15%, or alternatively +/−10%, or alternatively +/−5% or alternatively +/−2%. It is to be understood, although not always explicitly stated, that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a compound” includes a plurality of compounds, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this disclosure. Embodiments defined by each of these transition terms are within the scope of this disclosure.

In this document, the term self-microemulsifying drug delivery system (SMEDDS) is defined as a system that, upon dilution with an aqueous solution or phase, form microemulsions with sizes often ranging in the 1-200 nm range.

In this document, the term “fully dilutable SMEDDS” is defined as a system that, upon dilution with an aqueous solution or phase, would produce a single-phase microemulsion (μE), without excess phases (no liquid phase separation), no formation of precipitate and avoiding viscous (more than 1000 cP) liquid crystals, regardless of the aqueous solution content (from 0/100 of aqueous solution/SMEDDS to 99.99/0.001 of aqueous solution/SMEDDS).

The present disclosure relates to fully-dilutable SMEDDS compositions used to solubilize and deliver poorly water-soluble polar active ingredients via topical, transdermal, oral, transnasal, buccal, vaginal, subcutaneous, parenteral and ophthalmic routes, transepidermal delivery in plants and soft-bodied insects, and transmembrane delivery in microorganisms. The fully-dilutable characteristic of the SMEDDS presented herein is not disrupted by the addition of poorly water-soluble polar active compounds such as ibuprofen, cannabidiol, nonylphenol, eugenol and so forth. That is, the introduction of a polar oil does not induce a phase inversion of the surfactant into the oil.

In aspects, the fully-dilutable SMEDDS of the present disclosure comprises: (a) a lecithin compound; (b) a hydrophilic linker (HL) or a combination of two or more hydrophilic linkers (HLs), the HL or each of the HLs in the combination having one hydrocarbon group with at least 50% or more alkyl chain distribution between 6 to 10 carbon atoms and the HL or the combination of two or more HLs having a Cc of about −5 or more negative than about −5; and (c) a carrier oil. For clarity, in the case of a combination of two or more HLs, the combination has a Cc of about −5 or more negative of −5. In aspects, the fully-dilutable SMEDDS further comprises the poorly water-soluble polar active ingredient (one or more than one active ingredient may be included). In aspects, the fully dilutable SMEDDS comprises between 30 parts of a mixture of the lecithin and hydrophilic linker and 70 parts of the carrier oil (D30) and 90 parts of the mixture of lecithin and hydrophilic linker and 10 parts of the carrier oil (D90). In aspects, the fully dilutable in aqueous phase SMEDDS comprises between 40 parts of a mixture of the lecithin and hydrophilic linker and 60 parts of the carrier oil (D40) and 80 parts of the mixture of lecithin and hydrophilic linker and 20 parts of the carrier oil (D80). In aspects, the fully dilutable in aqueous phase SMEDDS is D30, D35, D40, D45, D50, D55, D60, D65, D70, D75, D80, D85, D90 or D95.

Defatted plant-based lecithin is combined with a special class of C6-C10 hydrophilic linker having an extreme hydrophilic nature, quantified by a characteristic curvature (Cc) being more negative than about −5 to produce the desired SMEDDS. The Cc specification for the hydrophilic linker was found to be surprisingly necessary as two hydrophilic linker products with the same nominal structure can have highly different Cc values. Another unexpected feature of the SMEDDS containing a polar oil active ingredient of the present disclosure is that they do not require (i.e., optional) the addition of a C10+ lipophilic linker to prevent the formation of lecithin liquid crystals with viscosities greater than 1000 cP, surfactant precipitation or gel formation as previously reported by Nouraei and Acosta [21]. Abdelkader et al. indicate the need to use glycerol monooleate (in the PECEOL product), which has been used as lipophilic linker, at a ratio of at least 1 part of PECEOL/1 part of lecithin to minimize the formation of insoluble phases [25]. The SMEDDS compositions disclosed herein do not require the inclusion of PECEOL or any lipophilic linker to avoid the formation of insoluble phases or slowly dissolving SMEDDS. The disclosed compositions comprise at least one part (by mass) of the extreme hydrophilic linker (Cc more negative than −5) for 1 part of lecithin. In aspects, the composition of the present disclosure comprises anywhere from one part (by mass) to 20 parts (by mass) of the extreme hydrophilic linker per one part (by mass) of lecithin. In aspects, the composition of the present disclosure comprises one part (by mass) of lecithin to 1, 2, 3, 4, 5, 6, 7, 8, 9, 10, 11, 12, 13, 14, 15, 16, 17, 18, 19 or 20 parts (by mass) of the extreme hydrophilic linker. In aspects, the composition of the present disclosure comprises no more than 20 parts (by mass) of the extreme hydrophilic linker to one part (by mass) of lecithin. Compositions of more than 20 parts of the extreme hydrophilic linker to 1 part of lecithin have insufficient capacity to solubilize the solvent oil.

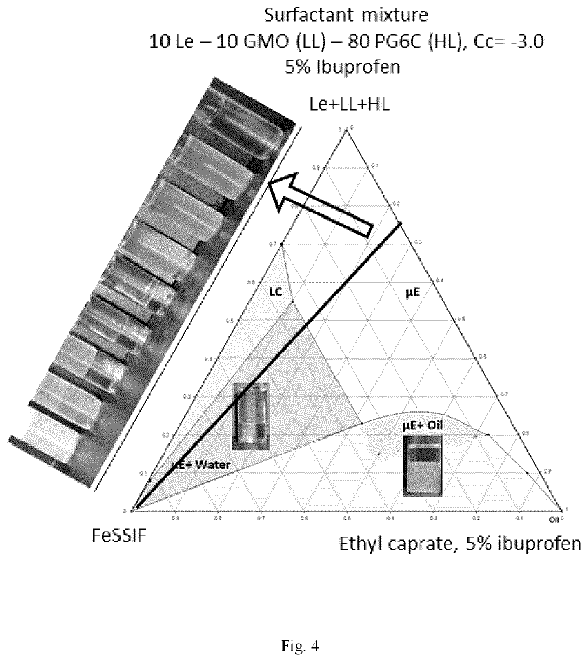

The complexity of formulating fully dilutable SMEDDS, even for compositions comprising lecithin and extreme hydrophilic linkers, is evidenced in the work of Sundar et al. [26]. In that work, mixtures of lecithin, lipophilic linker, and an extreme hydrophilic linker (Cc=−7.4) combined with hydrocarbons and diluted in water produced unstable emulsions (not microemulsions) with drop sizes ranging from 1 to 100 microns (1,000 nm to 100,000 nm). Sundar et al. used a weight fraction of lecithin+linkers in a mixture with the oil phase that was less than 10 wt %. As illustrated by the disclosed compositions shown in the ternary phase diagrams in FIGS. 5, 6, and 7, the weight fraction of lecithin+linkers in a mixture with a solvent oil required to produce microemulsions upon dilutions with aqueous solutions is very specific. This weight fraction is referred to as the dilution line “D” and often ranging from 30 to 90 wt % (D30 to D90) or from 40 to 80 wt % (D40 to D80). This range in dilution lines is the dilutability window. Systems with too little lecithin+linkers (under D30) or under D40) do not have enough surface-active material to solubilize all the solvent oil. Systems with too much lecithin+linkers (over D90 or over D80) produce viscous liquid crystals, with viscosities greater than 1000 cP, that are not dilutable within the typical dissolution test benchmark of 60 minutes applied to dilutable products [27]. The fully dilutable SMEDDS compositions herein disclosed produce single phases upon dilution with aqueous solutions (free or optional of aqueous co-solvents such as alcohols, glycerols or glycols) within 60 minutes of dilution and with minimal agitation (manual test tube rotation at 60) rotations/minute, for 5 minutes).

The SMEDDS compositions of lecithin with extreme hydrophilic linkers (Cc more negative than −5) containing polar oils were found to be fully dilutable in aqueous fed-state simulated intestinal fluid (FeSSIF or SIF), used as an example of a biological fluid. For dilutions containing between 20% and 80% of the aqueous phase (relevant to topical, transdermal, transnasal, buccal, vaginal and subcutaneous routes), the drop size of the system, measured via dynamic light scattering, was smaller than 10 nm. At dilutions between 80 to 99% aqueous phase, the size could grow up to 100 nm. Drop sizes of 10 nm and smaller are desirable for penetration through epidermal tissue and membranes [2]. However, even drops of 200 nm are still desirable for improved epithelial tissue uptake in drug delivery applications, including oral delivery [5].

### Lecithin

The formulation of lecithin linker microemulsions requires the use of a lecithin as a surfactant in the SMEDDS. A desirable feature of lecithin-based SMEDDS is that lecithin has generally recognized as safe (GRAS) status for food and pharmaceutical use. The term lecithin (including lysolecithin) refers to compounds or mixtures of phosphatidylcholines and other lipids and containing at least 50% w/w of a mixture of mono- and di-alkyl phosphatidylcholines, phosphatidylethanolamines, phosphatidylinositols and phosphatidylglycerols that can be obtained from animal (e.g., eggs), vegetable (e.g., soybean) sources or obtained through chemical synthesis. Preferred compositions are comprised of lecithin obtained from vegetable sources. Considering the minimum 1/20 lecithin to extreme hydrophilic linker ratio and the minimum D30 line, then the minimum lecithin content in the disclosed SMEDDS compositions is 1.5 wt %. Considering the maximum lecithin/extreme hydrophilic linker ratio of 1/1 and a maximum D90 line, then the maximum lecithin content in the disclosed SMEDDS is 45 wt %. Similarly, the minimum extreme hydrophilic linker content in lipophilic linker-free compositions is 10 wt %, and the maximum extreme hydrophilic linker content is 86 wt %. The term “lecithin” also includes synthetic-based phospholipid compounds. Non-limiting examples of synthetic-based phospholipid compounds that can be used as the surfactant in the SMEDDS includes stearamidopropyl PG-Dimonium Chloride Phosphate (and) Cetyl Alcohol. Arlasilk™ Phospholipid SV by Croda.

### Extreme Hydrophilic Linkers

The hydrophilic linkers used in this disclosure are amphiphilic, surfactant-like compounds containing 6 to 10 carbon atoms in their alkyl group and a Cc of about (i.e., +/−20%) −5 or more negative than about −5. Hydrophilic linkers used in the systems of the present disclosure are also referred to as extreme hydrophilic linkers. Hydrophilic linkers also include a mixture of compounds with the main (50% or more) alkyl chain distribution in between C6-C10 and the Cc of the combined mixture being about −5 or more negative than about −5. That is, the extreme hydrophilic linker can be combined with other extreme HLs or with a conventional hydrophilic linker (i.e., having a Cc less negative than −5+/−20%) to form a mixture, provided that the Cc of the mixture is about −5 or more negative than −5. For example, a mixture of compounds with the main (50% or more) alkyl chain distribution between C6-C10 having a combined Cc of −4.75 is an extreme hydrophilic linker. The hydrophilic group in these linkers can be anionic (sulfates, sulfonates, phosphates, phosphonates, carboxylates, sulfosuccinates) such as octanoates, octyl sulfonates, dibutyl sulfosuccinates: nonionic (carboxylic acids, alpha-hydroxy acids, esters of polyhydric alcohols, or glucosides, secondary ethoxylated alcohols, pyrrolidones) such as octanoic acid, 2-hydroxyoctanoic acid, hexyl and octyl polyglucosides, octyl pyrrolidone: cationic (amines, quaternary ammonium salts, amine oxides) such as octylamine: or zwitterionic (alkyl aminopropionic acids, betaines, sulfobetaines, phosphatidylcholines) such as octyl sulfobetaine, dibutyryl phosphatidylcholine, among others. Acosta et al. found that the short tail length of hydrophilic linkers, ranging between 6 and 10 carbons, and preferably between 6 and 9 carbons, reduces the interfacial rigidity of surfactant-oil-water (SOW) systems, including microemulsions, facilitating a quick solubilization process [28]. While the 6-10 carbon range in hydrophilic linkers helps avoid insoluble gel phases, the lecithin SMEDDS reported by Nouraei and Acosta produced with common hydrophilic linkers (Cc less negative than −5) still require the co-addition of a lipophilic linker to accomplish this feature. On the other hand, extreme hydrophilic linkers (Cc more negative than −5) can prevent the formation of insoluble lecithin phases (lecithin gels) even without a lipophilic linker. The difference between a conventional hydrophilic linker and an extreme hydrophilic linker is primarily observed via Cc values, obtained using the reference-test surfactant method of Zarate [22]. The Cc value is linked to the structure of the surfactant or linker molecule. Acosta et al. indicate that the presence of highly hydrophilic groups like ionic sulfate or sulfonate groups or numerous hydrogen-bonding groups produce negative shifts in the value of the Cc[29]. Extreme hydrophilic linkers contain multiple ionic groups or multiple hydrogen bonding groups in their headgroup. Sundar et al. reported a C8 extreme hydrophilic linker with a polyglycerol group containing—on average—10 glycerol units; this polyglyceryl-10-caprylate has a Cc=−7.4 [26,30]. However, the number of charges or hydrogen bonding groups in the surfactant headgroup is not a sufficient indicator for an extreme hydrophilic linker. For example, polyglycerol-6-caprylate produced by one surfactant manufacturer (Dermofeel® G6CY) has been reported to have a Cc=−3.0[30]. In example 1, the Cc determined for a polyglycerol-6-caprylate produced by a different surfactant manufacturer (Caprol® 6GC8) is determined to be Cc=−6.4. Example 3 illustrates that a composition comprising polyglycerol-6-caprylate by Dermofeel® G6CY is not fully dilutable in the presence of ibuprofen, a polar oil. Unexpectedly, replacing the polyglycerol-6-caprylate Dermofeel® G6CY with a polyglycerol-6-caprylate Caprol® 6GC8 results in a fully-dilutable SMEDDS (see Example 5). Replacing polyglycerol-6-caprylate Dermofeel® G6CY with polyglycerol-10-caprylate, with an extreme negative curvature of −7.4+/−1 (see table 15) also results in a fully-dilutable SMEDDS (example 4). Disodium C6-C10 glutamate is another example of an extreme hydrophilic linker.

The definition of an extreme hydrophilic linker, therefore, includes any molecule containing C6-C10 hydrocarbon chains, with multiple ionic groups (sulfates, sulfonates, benzene sulfonates, lignosulfonates, carboxylates, phosphates, phosphonates, polyphosphates, nitrates, quaternary ammonium groups, carbonates, sulfosuccinates, glutamates), multiple zwitterionic groups (betaines, phosphatidylcholines, peptides, polypeptides, hydrolyzed proteins, aminoxides), or multiple neutral hydrogen bonding groups (polyhydric alcohols, carbohydrate oligomers, polysaccharides, polyglycerols, polyglucosides, polyvinyl alcohol) producing a molecule with Cc of about −5 or more negative than about −5. The preferred hydrophilic linkers include polyglycerol esters of C6-C10 fatty acids, given their food additive status.

As previously described the term “about” as it relates to Cc includes a range of +/−20%. Therefore, a Cc of about −5 would include a Cc of −4, −4.1, −4.2, −4.3, −4.4, −4.5, −4.6, −4.7, −4.8, −4.9, −5 (i.e., −5+20% of −5).

Table 15 lists the Cc of selected biobased surfactants (adapted from [21]).

### Lipophilic Linker

The compositions disclosed herein do not require the use of a lipophilic linker. Lipophilic linkers generally refer to amphiphilic molecules with 11 or more carbon in the alkyl chain. Examples of lipophilic linkers include alcohols such as dodecyl alcohol, oleyl alcohol, cholesterol: fatty acids such as lauric acid, palmitic acid, oleic acid, omega 6-fatty acids, omega 3-fatty acids: fatty acid esters of sorbitol, maltitol, xylitol, isomalt, lactitol, erythritol, pentaerythritol, glycerol. Lipophilic linkers are reported to increase the interaction and solubilization capacity of the solvent oil [21]. In one of the embodiments, lipophilic linkers are included to improve the solvent oil solubilization capacity. The lipophilic linker to lecithin ratio in the disclosed compositions is 1/1. Considering the lipophilic linker/lecithin ratio of 1/1, the maximum lecithin/extreme hydrophilic linker ratio of 1/1, and a maximum D90 line, then the maximum lipophilic linker content in the disclosed SMEDDS is 30 wt %.

### Carrier Oil

The solvent or carrier oil facilitates the dissolution of the polar oil solute (i.e., the active ingredient) in the SMEDDS. The presence of solvent oil also hinders the formation of insoluble or slow-dissolving lecithin SMEDDS. On the other hand, too much solvent oil creates an emulsified excess oil phase upon the addition of water, which is incompatible with the idea of a fully dilutable SMEDDS. The ternary phase diagrams disclosed herein show that the SMEDDS dilutability window ranges from D40 (+/−10) to D80 (+/−10). Therefore, the solvent oil content in the disclosed compositions ranges from 10 wt % (at D90) to 70 wt % (at D30). The carrier oil can be a single solvent or a mixture of more than one solvent. Preferred solvent (carrier oil) includes alkyl esters of fatty acids such as isopropyl myristate, ethyl caprate, methyl oleate, ethyl oleate; terpenes such as limonene, pinene; and mixtures of with mono- di- and triglycerides used as cosolvents. In some cases, the solvent oil could be completely or partially substituted by a polar oil active, for example, vitamin E, ethyl esters or polyunsaturated fatty acids, or mixtures thereof.

### Polar Oil Active Compounds

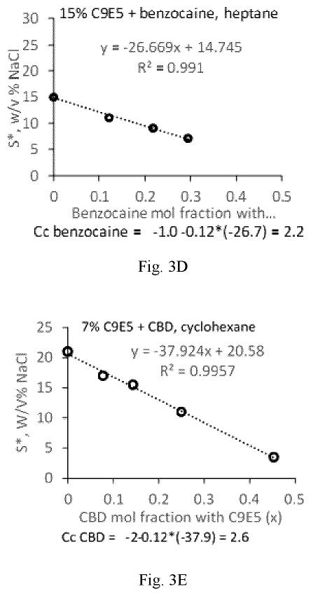

The SMEDDS compositions disclosed herein are specifically designed to deliver poorly soluble (in water) actives with a polar oil character. The limited aqueous solubility of these drugs prevents them from being molecularly dissolved at concentrations required to impart the desired effect in the aqueous environments of bodily fluids in animals, plant and insect fluids, or in aqueous environments containing microorganisms. The SMEDDS provide these drugs with an amphiphilic media that is fully dilutable in aqueous environments, producing microemulsion systems that contain the poorly soluble polar active ingredient in thermodynamic equilibrium. Lecithin-linker microemulsions formulated with conventional hydrophilic linkers (Cc less negative than −5) and containing poorly soluble polar actives, such as beta-sitosterol, have been disclosed [7]. However, ternary phase diagrams of those compositions reveal that they are not fully dilutable with aqueous solutions. Instead, the dilution of compositions comprising conventional hydrophilic linkers with intestinal fluid leads to the formation of unstable emulsions (as opposed to thermodynamically stable microemulsions) with drop sizes often ranging from 200 to 1000 nm. The lack of a fully dilutable path for lecithin and conventional hydrophilic linkers in systems containing a poorly soluble polar oil is also illustrated in Example 3 and FIG. 4 for a system containing ibuprofen as polar oil. The use of the disclosed compositions to achieve a fully dilutable path with ibuprofen and an extreme hydrophilic linker is shown in Example 4 and FIG. 5.

The special nature of polar oils has only been recently fully quantified using the hydrophilic-lipophilic-difference (HLD) framework [23,31]. According to that quantification, a polar oil can be partly considered to behave as a surfactant with positive characteristic curvature (Cc) and partly as an oil with a negative equivalent alkane carbon number (EACN). A positive Cc or a negative EACN leads to a positive shift in HLD, which is effectively compensated by the highly negative Cc value of the extreme hydrophilic linker.

“Poorly soluble oils” are defined as having an aqueous solubility of less than 1% w/w in isotonic solutions at room temperature and being soluble in the organic (carrier) solvents, according to U.S. Pat. No. 9,918,934. Ghayour and Acosta noted that polar oils are a broad class of oils consisting of heteroatom-linked polar groups attached to a nonpolar hydrocarbon group [23]. The disclosed compositions are comprised of polar oils containing a polar group, having an aqueous solubility lower than 1 wt %, logP of 1 or greater, having hydrogen bonding donors or hydrogen bonding acceptor groups, a non-zero dipole moment or a non-zero polar surface area, and a positive Cc or negative EACN, determined as per the method of Ghayour and Acosta, using a nonionic surfactant as a reference surfactant [23,31].

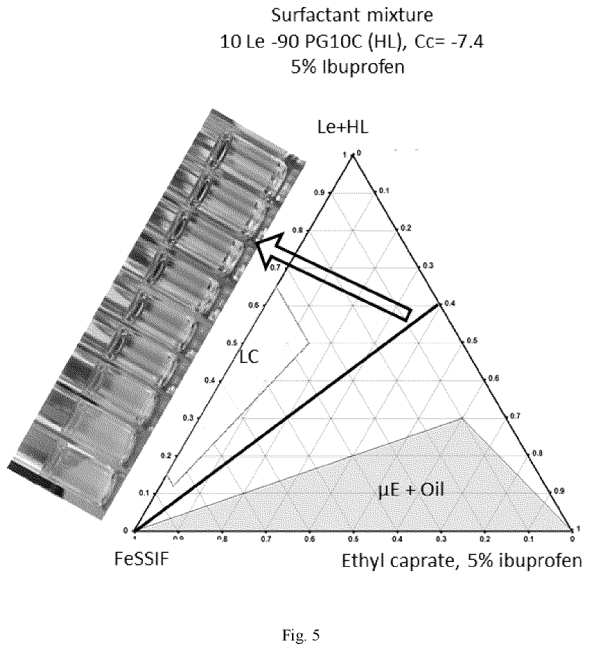

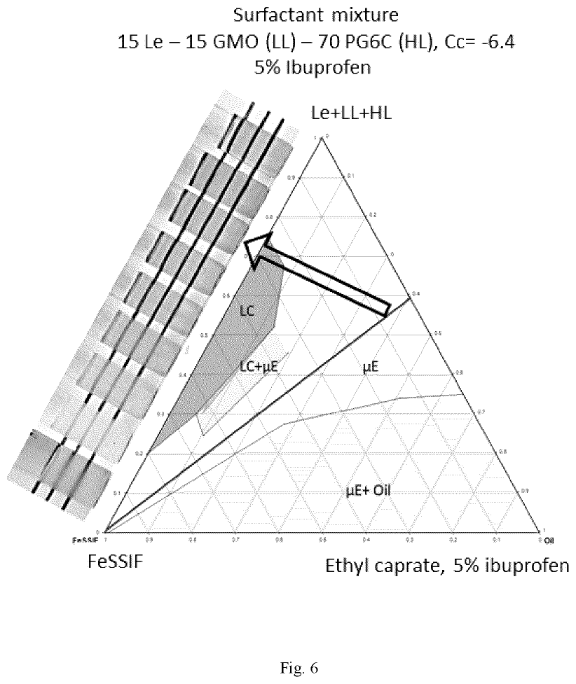

Example 2 illustrates the use of the method of Ghayour and Acosta to determine the Cc of ibuprofen, nonylphenol, eugenol, benzocaine, and cannabidiol (CBD) as example polar oils. Table 1 in Example 2 shows evidence that candidate polar oils with logP>1, having aqueous solubilities of less than 1 wt %, having non-zero polar areas or dipole moments, and hydrogen bonding donors or acceptors have a positive Cc value. Example 4 and FIG. 5 show disclosed compositions for SMEDDS containing ibuprofen. Example 5 and FIG. 6 show disclosed compositions for SMEDDS containing ibuprofen, extreme hydrophilic linker and glycerol monooleate (GMO) as a lipophilic linker. Examples 6 and 7 show disclosed compositions containing nonylphenol as an example of polar oil combined with an extreme hydrophilic linker. Examples 8 and 9 and FIG. 7 show disclosed compositions containing cannabidiol (CBD), as example polar oil. Example 12 presents a disclosed composition containing eugenol as polar oil and an extreme hydrophilic linker.

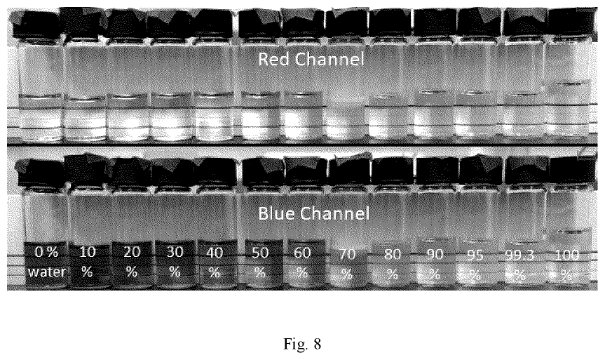

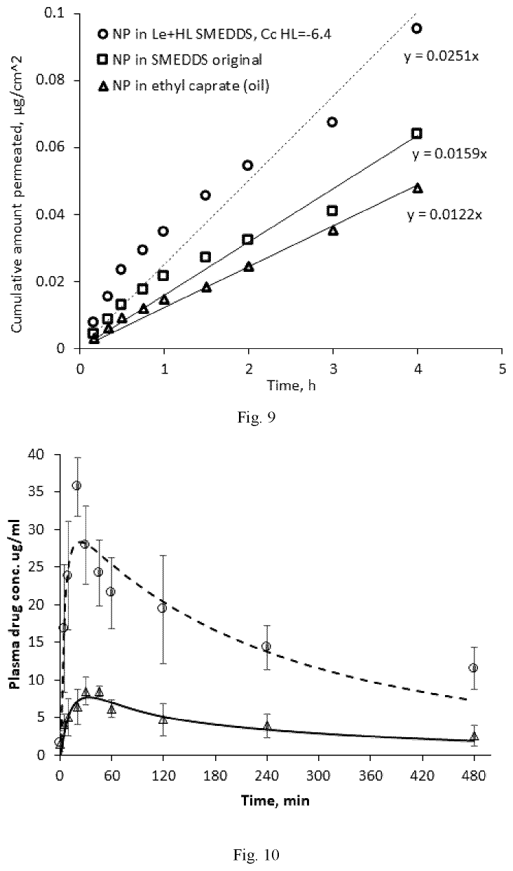

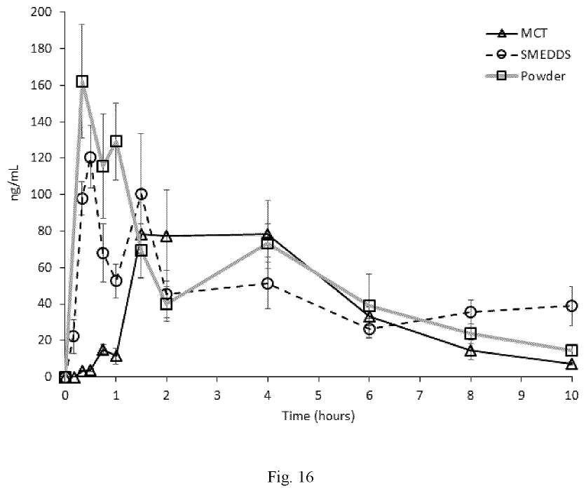

The polar oil actives can be used in a variety of applications, including but not limited to, nutritional or nutraceutical applications in humans and animals: the delivery of pharmaceutically active ingredients (API), including cannabinoids: as biocides or biostatic (preservatives) compounds in food, pharmaceutical, cosmetic, antiseptic, disinfectant, and agrochemical applications. Examples 10, 11 and 16, and FIGS. 9, 10 and 16 show the improved flux and delivery performance in transmembrane and oral delivery applications achieved with the disclosed SMEDDS compositions comprising an extreme hydrophilic linker and a polar oil (nonylphenol shown in FIG. 9, ibuprofen shown in FIG. 10, and CBD in FIG. 16). Example 16 also includes an encapsulated or powder version of the SMEDDS that provides a fast delivery of CBD, used as an example polar oil

Some polar oils, such as vitamin E and ethyl esters of polyunsaturated fatty acids, can also play the role of lipophilic linkers and oil solvents. The maximum polar oil content in a given composition can be estimated considering a D30 dilution line and a 1:1:1 ratio of lecithin:extreme hydrophilic linker:lipophilic linker. Under those conditions, the maximum polar oil content is 80 wt %.

The list of polar oils actives include, but is not limited to halogenated compounds such as fenbuconazole, Prochlorperazine, Triazolam, Fenchlorphos, Diazepam, Lorazepam, Griseofulvin, Chlorzoxazone, Metazachlor, Metolachlor, Dimethenamid, Lufenuron, Chlortoluron, Linuron, Metoxuron, Diuron, Diflubenzuron, Fluometuron, Chlorbromuron, Cyproconazole, Triticonazole, Triadimefon, Triadimenol, Tebuconazole, Propiconazole, Epoxiconazole, Prochloraz; long chain alcohols such as Lovastatin, Danazo, Equilin, Equilenin, Danthron, Estriol, alpha-tocopherol, Estradiol, Stanolone, Terfenadine, Dihydroequilenin, Norethisterone, Quinestrol, Quinidine, Haloperidol, Benperidol, Perphenazine, Simvastatin, Testosterone, Prasterone, Methyltestosterone, Estrone, Oxazepam, Pentazocin, Betamethasone, Triamcinolone, Dexamethasone, Abiraterone, Cortisone, Corticosterone, Prednisolone, Butylparaben, Hydrocortisone, Phenolphthalein, Quinidine, Quinine, Diosmetin, Propylparaben, prostaglandins, Ethylparaben, Atropine, Hyoscyamine, methylparaben, Butylated hydroxytoluene, Retinol, eugenol, linalool, Citronellol, terpenols, alkylphenols, para-aminophenol derivatives, terpenephenolics, Cannabidiol, Tetrahydrocannabinol, Cannabinol, Cannabigerols, Cannabichromenes: amines such as Clofazimine, Amitriptyline, Promethazine, Phenytoin, Tenoxicam, Indapamide, Bumetanide, Carbamazepine, Metoclopramide, Butamben, Heptabarbital, Oxamniquine, Reposal, Pentobarbital, Benzocaine, barbiturates, Phenacetin, Glutethimide, Chlordiazepoxide, Disopyramide, Simazine (6-chloro-N2,N4-diethyl-1,3,5-triazine-2,4-diamine), Atrazine (6-chloro-N2-ethyl-N4-isopropyl-1,3,5-triazine-2,4-diamine), Propazine (6-chloro-N,N′-bis(1-methylethyl)-1,3,5-triazine-2,4-diamine), Prometrine, Desmetrine, Terbutrine; acids such as Fenbufen, Diclofenac, Sulindac, Indoprofen, Indomethacin, Flufenamic acid, Iopanoic acid, Diflunisal, Naproxen, Ibuprofen, Mefenamic acid, Flurbiprofen, Nalidixic acid, Ketoprofen, Alclofenac, Diatrizoic acid, Salicylic acid, Aspirin, Benzoic acid, anthranilic acids, arylpropionic acids, enolic acids, heretoaryl acetic acids, indole and indene acetic acids, salicylic acid derivatives, nucleic acids, Phenoxyacetic acid, 2,4-dichlorophenoxyacetic acid, MCPA (2-(2,4-dichlorophenoxy)propanoic acid, 4-(2,4-dichlorophenoxy)butanoic acid). The disclosed compositions can be further comprised of mixtures of two or more polar oils.

The use of biological solutions and water as diluting media is a useful and distinctive feature of the disclosed formulation. Biologically relevant aqueous solutions such as FeSSIF, used in all disclosed examples except for Examples 9 and 12, make the disclosed compositions useful in pharmaceutical, cosmetic, food and agrochemical products. Examples 9 and 10 show that deionized water alone is also a suitable solvent for the dilution of the disclosed SMEDDS compositions. This is also a desirable feature for the disclosed SMEDDS, as these SMEDDS could be incorporated into clear liquids such as bottled or tap water, soft drinks, juices, energy drinks, and alcoholic beverages with less than 20% alcohol. Example 9 shows that intermediate dilutions can produce turbidity values close to 100 NTU, compatible with the turbidity of many fruit juices and milk-containing products and that at high dilutions (more than 95% aqueous phase), this turbidity can approach 0 NTU, close to that of clear drinks.

The disclosed SMEDDS compositions are water-dilutable, but they do not require the addition of water. Traces of water in the SMEDDS composition could be present due to the moisture in lecithin and in extreme hydrophilic linkers resulting from the manufacture, transport or storage of these ingredients.

An unexpected feature of the disclosed compositions is that introducing a relatively high concentration of polar oils, of 5 wt % or more in the SMEDDS, that serve as low molecular weight organogelators, can induce the formation of self-dispersing gels whose rate of dispersion can be controlled by the type and concentration of the organogelator. Polar oils that serve as low molecular weight organogelators include C12+ long-chain fatty acids such as 12-hydroxystearic acid (12-HSA) and stearic acid; long-chain fatty acid esters of polyhydric alcohols such as sorbitol monostearate (Span 60); long-chain amines such as Octadecanamide and (R)-12-hydroxyoctadecanamide; and sterol-based organogelators such as cholesterol, beta-sitosterol, gamma-oryzanol, and mixtures thereof.

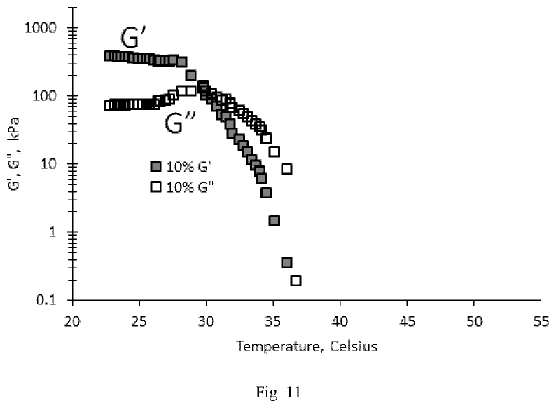

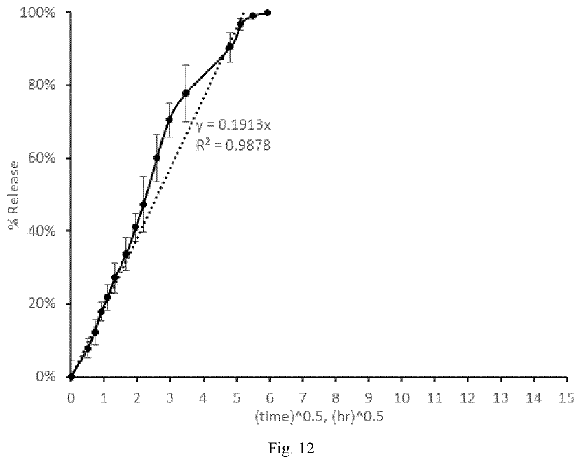

Example 13 discloses a D60 SMEDDS composition containing nonylphenol as model oil and 10% 12-HAS as organogelator. The rheological data for this gelled SMEDDS, shown in FIG. 11, indicates that this composition retains a gel-like structure until 30° C. When this gel was immersed in FeSSIF, the SMEDDS was fully diluted in the FeSSIF media. However, this release was not immediate (within 15 minutes) as it would normally happen in a SMEDDS dilution test. Instead, as evidenced in FIG. 12, a complete release took nearly one day. A slow-release is a desirable feature for SMEDDS compositions when the active ingredient is present in SMEDDS at high concentrations and whose immediate release could create undesirable side effects or unnecessary high concentrations of the active ingredient. A slow-release is also desirable to avoid frequent dosing, particularly when the dosing protocol requires complicated procedures such as subcutaneous injections or the surgical implantation of delivery devices.

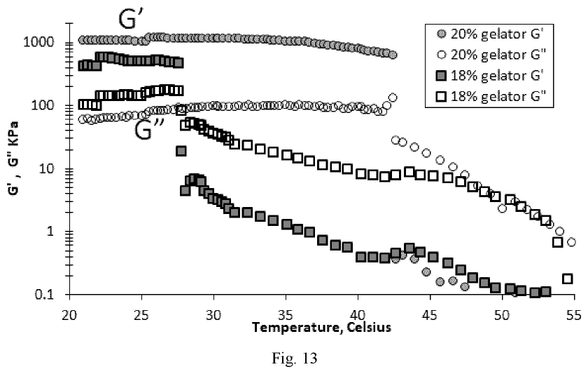

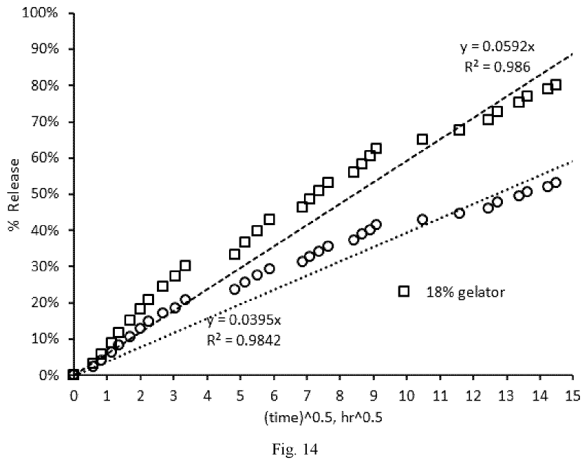

Example 14 presents another composition of gelled-SMEDDS, using a mixture of beta-sitosterol and gamma-oryzanol as organogelators and nonylphenol as model polar oil. Two concentrations of the mixture of organogelators were tested, 18 wt % and 20 wt %. The rheological properties for these systems are shown in FIG. 13, where the melting point for the 18 wt % organogelator system was found to be 28° C., and the melting point for the 20 wt % system was approximately 43° C. The release of the SMEDDS in these two organogel systems is presented in FIG. 14, where complete release from the 18 wt % system is expected after 12 days, and for the 20 wt % system is expected after 27 days.

The compositions disclosed in Examples 13 and 14 evidenced the tunable nature of the release profile, from hours to nearly a month, by adjusting the selection of the organogelator and its concentration in the SMEDDS composition.

The SMEDDS containing the polar oils of the present disclosure may be administered in the form of a tablet, granules, pellets or other multiparticulates, capsules, minitablets, beads, and as a powder, or any other suitable dosage form.

In another embodiment, solid encapsulated lecithin-linker SMEDDS containing polar oils are produced by combining the disclosed lecithin-linker SMEDDS containing polar oils with amphiphilic polymeric encapsulants having a glass transition temperature of less than 100° C. Having polymers with a low glass transition temperature, less than 100° C., allows for the spray drying encapsulation process at temperatures below 100° C. These low temperatures prevent the flash evaporation of the aqueous spray media, leading to more homogenous coating and the prevention of hot spots that could impact the quality of heat-sensitive polar oil solutes. The disclosed compositions could include polyacrylates or acrylate copolymers containing C2+ pendant hydrophobic groups that lend an amphiphilic nature to the polymer. The encapsulating polymer can also be obtained from natural sources such as shellac, a polyester resin of polyhydroxy carboxylic acids, and hydrophobically modified starches such as acetylated starches.

Example 15 discloses three encapsulated SMEDDS compositions, the first composition comprising a non-enteric polymer, EUDRAGUARD® (acetylated starch E1420), the second composition comprising an enteric EUDRAGIT L30 D-55 (methacrylic acid and ethyl acrylate copolymer), and the third composition comprising a PROTECT™ ENTERIC (shellac+sodium alginate) coating. The SMEDDS formulations contained nonylphenol as model polar oil. All the compositions were produced using a 40% SMEDDS loading, and feed spray drier temperatures of 70° C. All three encapsulated SMEDDS released more than half of the polar oil within one hour of exposure to FeSSIF. In acidic media, typical of gastric conditions, the encapsulating media hindered or preventing the release of the encapsulated SMEDDS. This pH-controlled release is useful to prevent the release of the polar oil solute in the stomach, which is a desirable feature in delivering active ingredients that can affect the lining of the stomach. The solids produced by the encapsulation process yielded flowable powders with resting angles near 30°, and particles ranging from 2 to 10 microns that make the powders amenable to integration into solid products, including flour, baked products, spices, and products compressed into pellets or tablet pills.

The following examples are intended to illustrate, but not limit the disclosure.

### EXAMPLES

### Example 1. Determination of the Characteristic Curvature (Cc) for Extreme Hydrophilic Linker Caprol® 6GC8 (Polyglycerol-6-Caprylate)

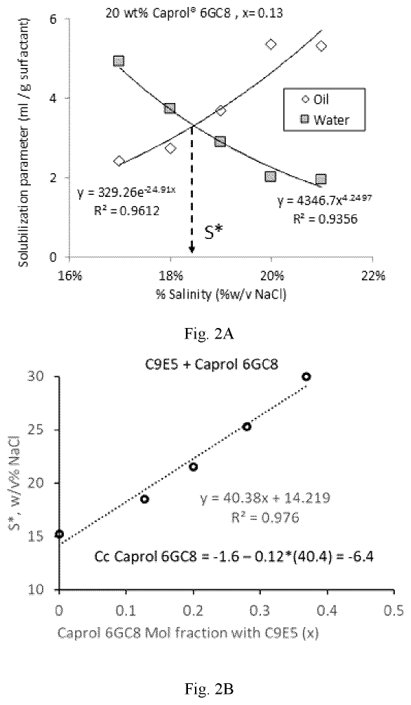

The characteristic curvature (Cc) of the candidate hydrophilic linker with extremely negative Cc (Cc<−5) was determined following the mixed reference and test surfactant method used by Zarate et al. [22]. In short, the test surfactant Caprol®) 6GC8 (polyglycerol-6-caprylate, molecular weight=593 g/mol) was mixed with a reference surfactant, Dehydol OD5® (C9E5, molecular weight=346 g/mol) with a calibrated Cc of −1.6 for a 10 wt % total surfactant concentration in the aqueous phase. For each mixture of Caprol® 6GC8 and the reference C9E5, the total surfactant concentration in the aqueous phase was maintained at 10 wt %. The salinity phase scans were conducted by vortex-mixing 2 mL of the aqueous surfactant solution, containing a set value of sodium chloride (g NaCl/100 mL of aqueous surfactant solution or % w/v NaCl) with 2 mL of n-heptane at room temperature in 2-dram vials sealed with a silicone-lined cap. The vials were mixed for 30 seconds twice a day for three days and then left to separate (equilibrate) for two weeks before reading the phase volumes of excess oil and water for systems that formed middle phase microemulsions. The difference between the initial volume added to the vial (2 mL), and the excess phase volume was considered the volume solubilized. The ratio between the volume solubilized (in mL) and the total mass of surfactant mixture (0.2 g) was reported as the solubilization parameter (SP) for the given phase at the set salinity. The graph of solubilization parameter for excess oil and water as a function of salinity was used to determine the optimal salinity (S*) of the system, where the SP for oil and water is the same [22]. FIG. 2A presents an example of the salinity phase scan SP curve for a system containing 20 parts (by mass) of the test surfactant Caprol® 6GC8 and 80 parts (by mass) of the reference surfactant C9E5.

Salinity scans for mixtures of Caprol® 6GC8:C9E5 were conducted for mass ratios 10:90, 20:80, 30:70, 40:60 and 50:50. The optimal salinity (S*) for every ratio was determined versus the molar fraction of test surfactant Caprol® 6GC8, and graphed as shown in FIG. 2B to determine the slope of the linear regression (dS*/dx=40.4% NaCl), and determine the Cc of the test surfactant, according to Zarate [22]:

$$Cc test surfactant = Cc reference surfactant - b \cdot ( {dS}^{*} / dx ) \qquad ( 2 )$$

Using the value of b=0.12 (% NaCl\hat{ }-1) for C9E5, according to Zarate et al. [22], then Cc Caprol®6GC8=−1.6−0.12(% NaCl\hat{ }-1)·(40.4% NaCl)=−6.4. This result highlights that, surprisingly, the nominal structure of a surfactant is not enough to indicate its characteristic curvature, considering that Dermofeel® G6CY, another polyglycerol-6-caprylate, has been found to have a Cc=−3 by Nouraei and Acosta who used the same methodology for Cc measurement. The reason for the large difference in Cc between Caprol® 6GC8 and Dermofeel® G6CY is unclear, but it is likely linked to the geometrical configuration of the headgroup, a factor not considered when citing a given surfactant or hydrophilic linker structure [32]. Another outstanding feature of Caprol®6GC8 having a Cc value of −6.4 is that there is only one additional hydrophilic linker reported in the literature with extreme negative curvature (Cc<−5), the polyglycerol-10-caprylate Polyaldo®10-1-CC, with Cc=−7.4 [30].

### Example 2. Determination of the Characteristic Curvature (Cc) for Poorly Water-Soluble Polar Active Ingredients

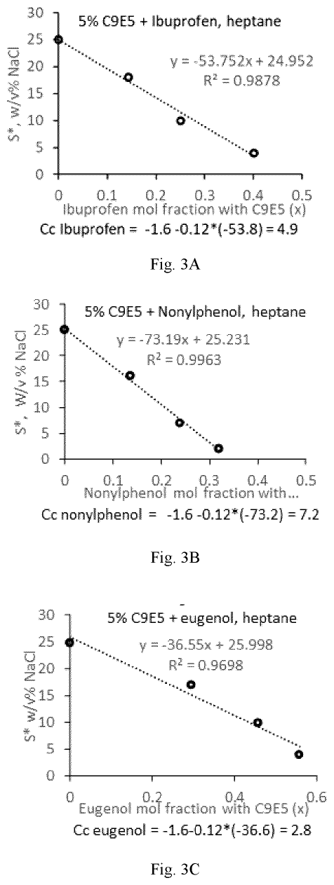

The definition of a poorly water-soluble active compounds having an aqueous solubility lower than 1 wt % in deionized water at room temperature follows that of U.S. Pat. No. 9,918,934. The definition of the polar nature of the active ingredient follows that of Ghayour and Acosta [23], where the polar oil at low concentrations, typically less than 30% in the system, behaves as a surfactant with a characteristic curvature determined via Equation 2, using the same salinity phase scan methodology described in Example 1. The Cc value reported for these polar oils is expected to be positive when using the procedure described in Example 1. Table 1 presents the dS*/dx values from FIG. 3 for the example polar oil active ingredients, ibuprofen (molecular weight 206 g/mol, Sigma-Aldrich ReagentPlus®, 99%), nonylphenol (molecular weight 220 g/mol, Sigma-Aldrich technical grade), cannabidiol (CBD, molecular weight 314 g/mol, The Valens Company, 96.3%), eugenol in clove oil (molecular weight 164 g/mol, NOW essentials, technical grade), and benzocaine (molecular weight 165 g/mol, Sigma-Aldrich 99%). As indicated by the values in Table 1, key factors required for the active are low water solubility (lower than 1 wt %), a relatively high logP (higher than 1), a non-zero dipole moment, and the presence of hydrogen bonding groups. These characteristics are likely to result in positive characteristic curvatures (Cc) typical of polar oils, as confirmed by the values in Table 1.

TABLE 1
Properties and calculated characteristic curvature of example poorly water-soluble
active ingredients for fully-dilutable lecithin-based SMEDDS. The aqueous solubility,
the negative logarithm of the dissociation constant (pKa), the logarithm of
the octanol-water partition constant (logP) and the number of hydrogen (H)
bonding donor and acceptor groups were obtained from the Drugbank database
for ibuprofen, eugenol, benzocaine and cannabidiol. For nonylphenol, the information
was obtained from the ChemSpider database, which was also used to obtain all
polar areas. Dipole moments obtained from Tantishaiyakul et al. [33]
The values of dS*/dx from FIG. 3 and Cc were obtained using the methodology
of Example 1, with C9E5 as reference surfactant.

| | | | | | Cannabidiol |
|---|---|---|---|---|---|
| Property | Ibuprofen | Nonylphenol | Eugenol | Benzocaine | (CBD) |
| Molecular weight | 206 | 220 | 164 | 165 | 314 |
| (g/mol) | | | | | |
| Aqueous solubility | 0.0021 | 0.0007 | 0.14 | 0.13 | 0.0013 |
| (wt %) | | | | | |
| pKa | 5.3 | Neutral | Neutral | 2.51 | Neutral |
| logP | 3.8 | 5.8 | 2.7 | 1.9 | 6.1 |
| Dipole moment (Debye) | 1.9 | 1.6 | 2.8 | 3.9 | 1.9 |
| Polar area (Å²) | 37 | 20 | 29 | 52 | 40 |
| Number of H-donors | 1 | 1 | 1 | 2 | 2 |
| Number of H-acceptors | 2 | 2 | 2 | 3 | 2 |
| dS*/dx (% w/v NaCl) | −54 | −73 | −37 | −27 | −38 |
| Cc | +4.9 | +7.2 | +2.8 | +1.6 | +2.6 |

### Example 3. Ibuprofen in SMEDDS with Conventional Hydrophilic Linker (Cc=−3)

In these formulations, soybean-extracted lecithin with Cc=+5.5 was used as the principal surfactant to produce the SMEDDS formulation. The hydrophilic linker used was a conventional hydrophilic linker, polyglycerol-6-caprylate, product name Dermofeel® G6CY, with Cc=−3.0. The lipophilic linker used was glycerol monooleate, product name Peceol™ with Cc=+6.6. The carrier (solvent) oil phase was ethyl caprate with EACN=+5.1. The SMEDDS was produced by first mixing 10 parts (by mass) of Lecithin with 10 parts of the lipophilic linker Peceol™ and 80 parts of the hydrophilic linker Dermofeel® G6CY using a vortex-mixer. A prescribed ratio of 25 parts (by mass) of ethyl caprate (carrier oil) and 75 parts of the Lecithin+linkers mixture was then mixed using a vortex-mixer. 95 parts (by mass) of the resulting mixture were then mixed with 5 parts of ibuprofen powder. The mixture was then vortex-mixed until no residual solids were observed in the liquid solution. The resulting solution was then diluted with fed-state simulated intestinal fluid (FeSSIF) at wt % ranging from 10 to 90. The diluted systems were vortex-mixed and then left to equilibrate for two weeks at room temperature before recording any phase separation. Phase separation was recorded based on visual observation of a separate layer of excess oil or water or the presence of drops visible to the naked eye (˜1 micron or larger).

TABLE 2
Composition, and number of phases obtained upon FeSSIF
dilution of the lecithin-based SMEDDS disclosed by Nouraei and
Acosta [21] containing 5% ibuprofen as example of polar active.
Columns (a) through (f) represent the weight percentage of (a)
lecithin; (b) lipophilic linker, glycerol monooleate (Peceol ™);
(c) hydrophilic linker polyglycerol-6-caprylate Dermofeel ®G6CY
(Cc = −3.0); (d) Fed-state simulated intestinal fluid (FeSSIF)
containing 0.57% w/v NaOH, 1.18% w/v NaCl, 0.86% w/v acetic acid,
0.83% w/v sodium taurocholate, and 0.28% w/v lecithin; (e) ibuprofen;
(f) is the carrier (solvent) oil, ethyl caprate. The system corresponds
to a dilution line D75, containing 75 parts of surfactant + linkers
mixture for every 25 parts of carrier oil (ethyl caprate).

| Dilution | %(a) | %(b) | %(c) | % (d) | %(e) | %(f) | Phases |
|---|---|---|---|---|---|---|---|
| SMEDDS | 7.13 | 7.13 | 57 | 0 | 5 | 23.74 | 1 |
| 10/90 | 6.41 | 6.41 | 51.3 | 10 | 4.5 | 21.38 | 1 |
| 20/80 | 5.7 | 5.7 | 45.6 | 20 | 4 | 19 | 1 |
| 30/70 | 4.99 | 4.99 | 39.9 | 30 | 3.5 | 16.62 | 1 |
| 40/60 | 4.28 | 4.28 | 34.2 | 40 | 3 | 14.24 | 2 |
| 50/50 | 3.56 | 3.56 | 28.5 | 50 | 2.5 | 11.88 | 2 |
| 60/40 | 2.85 | 2.85 | 22.8 | 60 | 2 | 9.5 | 2 |
| 70/30 | 2.14 | 2.14 | 17.1 | 70 | 1.5 | 7.12 | 2 |
| 80/20 | 1.43 | 1.43 | 11.4 | 80 | 1 | 4.74 | 2 |
| 90/10 | 0.71 | 0.71 | 5.7 | 90 | 0.5 | 2.38 | 2 |

Additional dilution tests were conducted using dilution lines D10, D20, D30, D40, D50, D60, D70, D80, and D90. The resulting phases after FeSSIF dilution were then recorded in the ternary phase of FIG. 4. As indicated by the data in Table 2 and FIG. 4, the formulation with conventional hydrophilic linker Dermofeel® G6CY (Cc=−3.0) is a SMEDDS formulation because it can form microemulsions with the addition of up to 30% of the aqueous phase (dilution 30/70), but it is not a fully-dilutable SMEDDS because higher FeSSIF content produced phase separation.

### Example 4. Ibuprofen in Fully-Dilutable SMEDDS with an Extreme Hydrophilic Linker

SMEDDS were formulated with soybean-extracted lecithin (Cc=+5.5) as the principal surfactant, combined with an extreme hydrophilic linker, polyglycerol-10-caprylate, Polyaldo®10-1-CC (Cc=−7.4). The formulation was free of a lipophilic linker. The solvent oil phase was ethyl caprate with EACN=+5.1 [21]. The SMEDDS was produced by first mixing 10 parts (by mass) of Lecithin with 90 parts of the hydrophilic linker Polyaldo® 10-1-CC using a vortex-mixer. A prescribed ratio of 40 parts (by mass) of ethyl caprate (carrier oil) and 60 parts of the Lecithin+hydrophilic linker mixture was then mixed using a vortex-mixer. 95 parts (by mass) of the resulting mixture were then mixed with 5 parts of ibuprofen powder. The mixture was then vortex-mixed until no residual solids were observed in the liquid solution. The resulting solution was then diluted with fed-state simulated intestinal fluid (FeSSIF) at wt % ranging from 10 to 99. The diluted systems were vortex-mixed and then left to equilibrate for two weeks at room temperature before recording any phase separation. Phase separation was recorded based on visual observation of a separate layer of excess oil or water or the presence of drops visible to the naked eye (˜1 micron or larger). The viscosity of the formulation was determined via A Carri-Med CSL2 Rheometer (TA Instruments, New Castle, DE, USA) at 25° C., averaging the values obtained at shear rates ranging from 10 to 100 l/s. A Brookhaven (Holtsville, NY, USA) BI90 PLUS Particle Size Analyser was used to measure the drop size of the diluted microemulsions via photocorrelation spectroscopy of a 90°-scattered 635 nm laser beam. The data in Table 3 summarize the observations with the D60 dilution line (60 parts surfactant mixture, 40 parts carrier oil), indicating that a single-phase (full dilutability) was obtained with 10% to 99% FeSSIF as the diluting aqueous phase. These results illustrate the fully-dilutable nature of the SMEDDS produced with the extreme hydrophilic linker Polyaldo® 10-1-CC (Cc=−7.4). After repeating the dilution experiment for dilution lines D10, D30, D50, D70, and D90, the ternary phase diagram of FIG. 5 was produced. As per the diagram, for dilution lines between D40 to D70, there is a path of full dilutability.

TABLE 3
Composition, number of phases obtained upon FeSSIF dilution, viscosity and drop
size of lipophilic linker-free, lecithin-based SMEDDS prepared with the extreme
hydrophilic linker Polyaldo ®10-1-CC and 5% ibuprofen. Columns (a) through
(f) represent the weight percentage of (a) lecithin; (b) Peceol ™; (c)
extreme hydrophilic linker Polyaldo ®10-1-CC (Cc = −7.4); (d)
FeSSIF; (e) ibuprofen; (f) ethyl caprate. The system corresponds to a dilution
line D60, containing 60 parts of surfactant + hydrophilic linker mixture
for every 40 parts of carrier oil (ethyl caprate).

| | | | | | | | | Viscosity | Size |
|---|---|---|---|---|---|---|---|---|---|
| Dilution | %(a) | %(b) | %(c) | % (d) | %(e) | %(f) | Phases | cP | nm |
| SMEDDS | 5.7 | 0 | 51.3 | 0 | 5 | 38 | 1 | | |
| 10/90 | 5.13 | 0 | 46.17 | 10 | 4.5 | 34.2 | 1 | 100 ± 20 | 19 ± 2 |
| 20/80 | 4.56 | 0 | 41.04 | 20 | 4 | 30.4 | 1 | 80 ± 20 | 38 ± 4 |
| 30/70 | 3.99 | 0 | 35.91 | 30 | 3.5 | 26.6 | 1 | 70 ± 10 | 8 ± 2 |
| 40/60 | 3.42 | 0 | 30.78 | 40 | 3 | 22.8 | 1 | 40 ± 10 | 2 ± 1 |
| 50/50 | 2.85 | 0 | 25.65 | 50 | 2.5 | 19 | 1 | 30 ± 10 | 2 ± 1 |
| 60/40 | 2.28 | 0 | 20.52 | 60 | 2 | 15.2 | 1 | 25 ± 5 | 3 ± 1 |
| 70/30 | 1.71 | 0 | 15.39 | 70 | 1.5 | 11.4 | 1 | 20 ± 5 | 2 ± 1 |
| 80/20 | 1.14 | 0 | 10.26 | 80 | 1 | 7.6 | 1 | 15 ± 2 | 14 ± 2 |
| 90/10 | 0.57 | 0 | 5.13 | 90 | 0.5 | 3.8 | 1 | 4 ± 2 | 21 ± 2 |
| 99/1 | 0.057 | 0 | 0.513 | 99 | 0.05 | 0.38 | 1 | 1 ± 1 | 110 ± 10 |
| 99.9/0.1 | 0.006 | | 0.051 | 99.9 | 0.005 | 0.038 | 1 | 1 ± 1 | 50 ± 10 |

### Example 5. Ibuprofen in SMEDDS with Lipophilic and Extreme Hydrophilic Linkers

Fully-dilutable SMEDDS were formulated with soybean-extracted lecithin (Cc-+5.5) as the principal surfactant, extreme hydrophilic linker, polyglycerol-6-caprylate, Caprol® 6GC8 (Cc=−6.4), and lipophilic linker glycerol monooleate (Peceol™, Cc=+6.6). The carrier (solvent) oil phase was ethyl caprate. The SMEDDS was produced by first mixing 15 parts (by mass) of Lecithin with 15 parts of the lipophilic linker (Peceol™) and 70 parts of Caprol® 6GC8 using a vortex-mixer. A prescribed ratio of 40 parts (by mass) of ethyl caprate (carrier oil) and 60 parts of the Lecithin+linkers mixture was then mixed using a vortex-mixer (D60 composition). 95 parts (by mass) of the resulting mixture were then mixed with 5 parts of ibuprofen powder. The mixture was then vortex-mixed until no residual solids were observed in the liquid solution. The resulting solution was then diluted with fed-state simulated intestinal fluid (FeSSIF).

TABLE 4
Composition, number of phases upon FeSSIF dilution, viscosity, and drop
size of lipophilic linker-free, lecithin-based SMEDDS prepared with the
extreme hydrophilic linker Caprol ®6GC8, lipophilic linker Peceol ™ and
5% ibuprofen. Columns (a) through (f) represent the weight percentage
of (a) lecithin; (b) lipophilic linker; (c) extreme hydrophilic linker
polyglycerol-6-caprylate Caprol ®6GC8 with Cc = −6.4; (d)
FeSSIF; (e) ibuprofen; (f) ethyl caprate. The system corresponds to a
dilution line D60, containing 60 parts of surfactant + hydrophilic
linker mixture and 40 parts of carrier oil (ethyl caprate).

| | | | | | | | | Viscosity | Size |
|---|---|---|---|---|---|---|---|---|---|
| Dilution | %(a) | %(b) | %(c) | % (d) | %(e) | %(f) | Phases | cP | nm |
| SMEDDS | 8.6 | 8.6 | 39.9 | 0 | 5 | 38 | 1 | N.D. | N.D. |
| 10/90 | 7.7 | 7.7 | 35.9 | 10 | 4.5 | 34.2 | 1 | 170 ± 20 | 47 ± 10 |
| 20/80 | 6.8 | 6.8 | 31.9 | 20 | 4 | 30.4 | 1 | 85 ± 10 | 7 ± 1 |
| 30/70 | 6.0 | 6.0 | 27.9 | 30 | 3.5 | 26.6 | 1 | 80 ± 10 | 7 ± 1 |
| 40/60 | 5.1 | 5.1 | 23.9 | 40 | 3 | 22.8 | 1 | 70 ± 10 | 4 ± 1 |
| 50/50 | 4.3 | 4.3 | 20.0 | 50 | 2.5 | 19 | 1 | 60 ± 10 | 3 ± 1 |
| 60/40 | 3.4 | 3.4 | 16.0 | 60 | 2 | 15.2 | 1 | 40 ± 5 | 6 ± 1 |
| 70/30 | 2.6 | 2.6 | 12.0 | 70 | 1.5 | 11.4 | 1 | 20 ± 5 | 6 ± 1 |
| 80/20 | 1.7 | 1.7 | 8.0 | 80 | 1 | 7.6 | 1 | 15 ± 2 | 8 ± 1 |
| 90/10 | 0.9 | 0.9 | 4.0 | 90 | 0.5 | 3.8 | 1 | 4 ± 1 | 30 ± 3 |

N.D.: not determined

Additional dilution lines D10, D20, D30, D40, D50, D70, D80 and D90 were also evaluated using the same procedure used to produce the dilution line D60 presented in Table 4. The results from these studies are summarized in the ternary phase diagram of FIG. 6. According to the phases outlined in FIG. 6, a full dilutable path was obtained between D50 and D70.

The composition of Example 3 is similar to that of Example 5, with two differences, first that Example 3 had more hydrophilic linker (80 parts) as compared to Example 5 (70 parts). The second difference is that although both examples used a hydrophilic linker with the nominal structure of polyglycerol-6-caprylate, the Dermofeel® G6CY product used in Example 3 had a Cc=−3 and the Caprol®6GC8 product used in Example 5 was an extreme hydrophilic linker with Cc=−6.4. While having more hydrophilic linker should have helped Example 3 compensate for the presence of polar active (ibuprofen), it was the use of an extreme hydrophilic linker (Cc more negative than −5) what allowed the compositions of Example 5 obtain a fully-dilutable path.

### Example 6. Nonylphenol in SMEDDS with extreme hydrophilic linker (Cc=−7.4)

The SMEDDS was produced by first mixing 10 parts (by mass) of lecithin with 90 parts of the hydrophilic linker Polyaldo®10-1-CC using a vortex-mixer. A prescribed ratio of 40 parts (by mass) of ethyl caprate and 60 parts of the Lecithin+hydrophilic linker mixture was then mixed using a vortex-mixer. 90 parts (by mass) of the resulting mixture were then mixed with 10 parts of nonylphenol used as model polar oil. The resulting solution was diluted with FeSSIF. The diluted systems were vortex-mixed and then left to equilibrate for two weeks at room temperature.

TABLE 5
Composition, number of phases upon FeSSIF dilution,
viscosity, and drop size of lipophilic linker-free,
lecithin-based SMEDDS prepared with the extreme hydrophilic
linker Polyaldo ®10-1-CC and containing 10% Nonylphenol.
Columns (a) through (f) are weight percentage of (a)
lecithin; (b) lipophilic linker; (c) extreme hydrophilic
linker Polyaldo ®10-1-CC (Cc = −7.4); (d) FeSSIF;
(e) nonylphenol; (f) ethyl caprate. The system corresponds
to a dilution line D60, containing 60 parts of
surfactant + hydrophilic linker mixture for every
40 parts of carrier oil (ethyl caprate).

| | | | | | | | | Viscosity | Size |
|---|---|---|---|---|---|---|---|---|---|
| Dilution | %(a) | %(b) | %(c) | % (d) | %(e) | %(f) | Phases | cP | nm |
| SMEDDS | 5.4 | 0.0 | 48.6 | 0 | 10 | 36 | 1 | N.D. | N.D |
| 10/90 | 4.9 | 0.0 | 43.7 | 10 | 9 | 32.4 | 1 | 90 ± 10 | 2 ± 1 |
| 20/80 | 4.3 | 0.0 | 38.9 | 20 | 8 | 28.8 | 1 | 60 ± 20 | 2 ± 1 |
| 30/70 | 3.8 | 0.0 | 34.0 | 30 | 7 | 25.2 | 1 | 70 ± 10 | 4 ± 1 |
| 40/60 | 3.2 | 0.0 | 29.2 | 40 | 6 | 21.6 | 1 | 30 ± 5 | 4 ± 1 |
| 50/50 | 2.7 | 0.0 | 24.3 | 50 | 5 | 18 | 1 | 25 ± 5 | 3 ± 1 |
| 60/40 | 2.2 | 0.0 | 19.4 | 60 | 4 | 14.4 | 1 | 20 ± 2 | 5 ± 1 |
| 70/30 | 1.6 | 0.0 | 14.6 | 70 | 3 | 10.8 | 1 | 16 ± 2 | 4 ± 1 |
| 80/20 | 1.1 | 0.0 | 9.7 | 80 | 2 | 7.2 | 1 | 14 ± 2 | 3 ± 1 |
| 90/10 | 0.5 | 0.0 | 4.9 | 90 | 1 | 3.6 | 1 | 5 ± 2 | 40 ± 10 |
| 99/1 | 0.05 | 0.00 | 0.49 | 99.00 | 0.10 | 0.36 | 1 | 1 ± 1 | 100 ± 10 |

The existence of a single-phase throughout the entire dilution of the D60 composition in Table 5 confirms the fully-dilutable nature of the D60 composition containing 10% nonylphenol. This D60 composition is the same as that used for 5% ibuprofen in Example 4, further confirming the suitability of the lipophilic linker-free formulations to produce fully-dilutable formulations with a range of polar actives.

### Example 7. Nonylphenol in SMEDDS with Extreme Hydrophilic Linker and Limonene

The SMEDDS was produced by first mixing 10 parts (by mass) of lecithin with 90 parts of the hydrophilic linker Polyaldo®10-1-CC using a vortex-mixer. A prescribed ratio of 40 parts (by mass) of limonene (racemic mixture, technical grade) and 60 parts of the Lecithin+hydrophilic linker mixture was then mixed using a vortex-mixer. 95 parts (by mass) of the resulting mixture were then mixed with 5 parts of nonylphenol used as model polar oil. The resulting solution was diluted with FeSSIF. The diluted systems were vortex-mixed and then left to equilibrate for two weeks at room temperature.

TABLE 6
Composition, number of phases obtained upon FeSSIF dilution, viscosity and drop size
of lipophilic linker-free, lecithin-based SMEDDS prepared with the extreme hydrophilic
linker Polyaldo ®10-1-CC and 5% Nonylphenol. Columns (a) through (f) are
weight percentage of (a) lecithin; (b) lipophilic linker; (c) extreme hydrophilic
linker Polyaldo ®10-1-CC with Cc = −7.4; (d) FeSSIF; (e) nonylphenol;
(f) limonene. The system corresponds to a dilution line D60, containing 60 parts
of surfactant + hydrophilic linker, mixture for every 40 parts of carrier oil (limonene).

| | | | | | | | | Viscosity | Size |
|---|---|---|---|---|---|---|---|---|---|
| Dilution | %(a) | %(b) | %(c) | % (d) | %(e) | %(f) | Phases | cP | nm |
| SMEDDS | 5.7 | 0.0 | 51.3 | 0 | 5 | 38 | 1 | N.D. | N.D |
| 10/90 | 5.1 | 0.0 | 46.2 | 10 | 4.5 | 34.2 | 1 | 240 ± 20 | 2 ± 1 |
| 20/80 | 4.6 | 0.0 | 41.0 | 20 | 4 | 30.4 | 1 | 170 ± 20 | 2 ± 1 |
| 30/70 | 4.0 | 0.0 | 35.9 | 30 | 3.5 | 26.6 | 1 | 110 ± 10 | 3 ± 1 |
| 40/60 | 3.4 | 0.0 | 30.8 | 40 | 3 | 22.8 | 1 | 80 ± 10 | 5 ± 1 |
| 50/50 | 2.9 | 0.0 | 25.7 | 50 | 2.5 | 19 | 1 | 60 ± 5 | 8 ± 2 |
| 60/40 | 2.3 | 0.0 | 20.5 | 60 | 2 | 15.2 | 1 | 50 ± 5 | 6 ± 2 |
| 70/30 | 1.7 | 0.0 | 15.4 | 70 | 1.5 | 11.4 | 1 | 25 ± 2 | 5 ± 2 |
| 80/20 | 1.1 | 0.0 | 10.3 | 80 | 1 | 7.6 | 1 | 7 ± 2 | 23 ± 3 |
| 90/10 | 0.6 | 0.0 | 5.1 | 90 | 0.5 | 3.8 | 1 | 2 ± 2 | 110 ± 20 |
| 99/1 | 0.06 | 0.00 | 0.51 | 99.0 | 0.05 | 0.38 | 1 | 1 ± 1 | 130 ± 20 |
| 99.9/0.1 | 0.006 | 0.000 | 0.051 | 99.90 | 0.005 | 0.038 | 1 | 1 ± 1 | 36 ± 4 |

The data in Table 6 confirm the fully-dilutable nature of the D60 composition containing 5% nonylphenol and limonene as the carrier or solvent oil. This D60 composition is the same as that of Example 6 except that ethyl caprate was substituted for a terpene, limonene, exemplifying the variety of carrier (solvent) oils that can be used.

### Example 8. Cannabidiol (CBD) in SMEDDS with an Extreme Hydrophilic Linker

The SMEDDS was produced by first mixing 10 parts (by mass) of lecithin with 90 parts of the hydrophilic linker Polyaldo®10-1-CC using a vortex-mixer. A prescribed ratio of 40 parts (by mass) of limonene (racemic mixture, technical grade) and 60 parts of the Lecithin+hydrophilic linker mixture was then mixed using a vortex-mixer. 95 parts (by mass) of the resulting mixture were then mixed with 5 parts of CBD used as model polar oil. The resulting solution was diluted with FeSSIF. The diluted systems were vortex-mixed and then left to equilibrate for two weeks at room temperature.

TABLE 7
Composition, number of phases obtained upon FeSSIF dilution, viscosity (from
Example 7-same SMEDDS, different drug), and drop size of lipophilic linker-free,
lecithin-based SMEDDS prepared with the extreme hydrophilic linker Polyaldo ®10-1-CC
and 5% CBD. Columns (a) through (f) are weight percentage of (a) lecithin; (b)
lipophilic linker; (c) extreme hydrophilic linker Polyaldo ®10-1-CC with
Cc = −7.4; (d) FeSSIF; (e) CBD; (f) limonene. The system corresponds
to a dilution line D60, containing 60 parts of surfactant + hydrophilic
linker mixture for every 40 parts of carrier oil (limonene).

| | | | | | | | | Viscosity | Size |
|---|---|---|---|---|---|---|---|---|---|
| Dilution | %(a) | %(b) | %(c) | % (d) | %(e) | %(f) | Phases | cP | nm |
| SMEDDS | 5.7 | 0.0 | 51.3 | 0 | 5 | 38 | 1 | N.D. | N.D |
| 10/90 | 5.1 | 0.0 | 46.2 | 10 | 4.5 | 34.2 | 1 | 240 ± 20 | 3.5 ± 2 |
| 20/80 | 4.6 | 0.0 | 41.0 | 20 | 4 | 30.4 | 1 | 170 ± 20 | 4.0 ± 2 |
| 30/70 | 4.0 | 0.0 | 35.9 | 30 | 3.5 | 26.6 | 1 | 110 ± 10 | 1.5 ± 2 |
| 40/60 | 3.4 | 0.0 | 30.8 | 40 | 3 | 22.8 | 1 | 80 ± 10 | 6.0 ± 2 |
| 50/50 | 2.9 | 0.0 | 25.7 | 50 | 2.5 | 19 | 1 | 60 ± 5 | 7.5 ± 3 |
| 60/40 | 2.3 | 0.0 | 20.5 | 60 | 2 | 15.2 | 1 | 50 ± 5 | 3.0 ± 2 |
| 70/30 | 1.7 | 0.0 | 15.4 | 70 | 1.5 | 11.4 | 1 | 25 ± 2 | 2.5 ± 2 |
| 80/20 | 1.1 | 0.0 | 10.3 | 80 | 1 | 7.6 | 1 | 7 ± 2 | 35 ± 9 |
| 90/10 | 0.6 | 0.0 | 5.1 | 90 | 0.5 | 3.8 | 1 | 2 ± 2 | 35 ± 10 |
| 95/5 | 0.06 | 0.00 | 0.51 | 99.0 | 0.05 | 0.38 | 1 | 1 ± 1 | 40 ± 13 |
| 99/1 | 0.006 | 0.000 | 0.051 | 99.90 | 0.005 | 0.038 | 1 | 1 ± 1 | 90 ± 10 |

Additional dilution lines DO, D10, D20, D30, D40, D50, D70, D80, D90 and D100 were evaluated; the results from these studies are summarized in the ternary phase diagram of FIG. 7. According to the phases outlined in FIG. 7, a fully dilutable path was obtained between D45 and D60.

### Example 9. Cannabidiol (CBD) in SMEDDS Diluted in Distilled Water

The SMEDDS was produced by first mixing 10 parts (by mass) of lecithin with 90 parts of hydrophilic linker Polyaldo®10-1-CC using a vortex-mixer. A prescribed ratio of 30 parts of limonene and 70 parts of Lecithin+hydrophilic linker mixture was then mixed using a vortex-mixer. 95 parts (by mass) of the resulting mixture were then mixed with 5 parts of CBD used as model polar oil. The resulting solution was diluted with distilled water. The diluted systems were vortex-mixed and then left to equilibrate for two hours at room temperature before taking a picture of the system for image analysis.

TABLE 8
Composition, number of phases obtained upon distilled water dilution, light
attenuation coefficient (Kd, m⁻¹) and estimated turbidity (NTU) of lecithin-
based SMEDDS prepared with the extreme hydrophilic linker Polyaldo ®10-1-CC
and 5% CBD. Columns (a) through (f) are weight percentage of (a) lecithin;
(b) lipophilic linker; (c) extreme hydrophilic linker Polyaldo ®10-1-CC
with Cc = −7.4; (d) distilled water; (e) CBD; (f) limonene. The system
corresponds to a dilution line D70, containing 70 parts of surfactant +
hydrophilic linker mixture for every 30 parts of carrier oil (limonene).

| Dilution | %(a) | %(b) | %(c) | % (d) | %(e) | %(f) | Phases | Kd, m⁻¹ | NTU |
|---|---|---|---|---|---|---|---|---|---|
| SMEDDS | 6.65 | 0 | 59.85 | 0 | 5 | 28.5 | 1 | 0 ± 1 | 0 ± 4 |
| 10/90 | 5.99 | 0 | 53.87 | 10 | 4.5 | 25.65 | 1 | 0 ± 1 | 0 ± 4 |
| 20/80 | 5.32 | 0 | 47.88 | 20 | 4 | 22.8 | 1 | 3 ± 1 | 12 ± 4 |
| 30/70 | 4.66 | 0 | 41.90 | 30 | 3.5 | 19.95 | 1 | 0 ± 1 | 0 ± 4 |
| 40/60 | 3.99 | 0 | 35.91 | 40 | 3 | 17.1 | 1 | 0 ± 1 | 0 ± 4 |
| 50/50 | 3.33 | 0 | 29.93 | 50 | 2.5 | 14.25 | 1 | 5 ± 1 | 20 ± 4 |
| 60/40 | 2.66 | 0 | 23.94 | 60 | 2 | 11.4 | 1 | 7 ± 1 | 28 ± 4 |
| 70/30 | 2.00 | 0 | 17.96 | 70 | 1.5 | 8.55 | 1 | 21 ± 1 | 84 ± 4 |
| 80/20 | 1.33 | 0 | 11.97 | 80 | 1 | 5.7 | 1 | 27 ± 1 | 108 ± 4 |
| 90/10 | 0.67 | 0 | 5.99 | 90 | 0.5 | 2.85 | 1 | 12 ± 1 | 48 ± 4 |
| 95/5 | 0.33 | 0 | 2.99 | 95 | 0.25 | 1.43 | 1 | 16 ± 1 | 64 ± 4 |
| 99.3/0.7 | 0.047 | 0 | 0.42 | 99.3 | 0.035 | 0.20 | 1 | 1 ± 1 | 4 ± 4 |

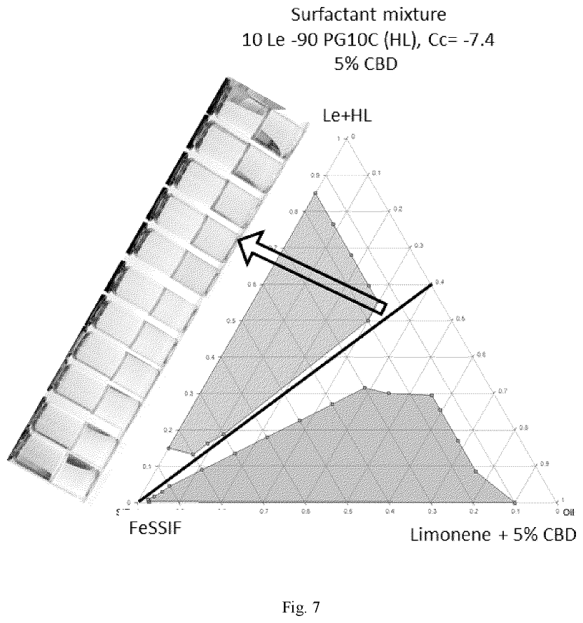

The attenuation, Kd=1/light path length*ln(transmittance through water/transmittance through the sample), was based on transmittance estimated using image-J analysis of grey levels in the red channel of the test tubes in FIG. 8. The NTU turbidity of the samples was estimated as 4*Kd, based on approximate literature correlations[34].

### Example 10. Transdermal Permeation of Nonylphenol Formulated in SMEDDS with Extreme Hydrophilic Linker (Cc=−7.4)

SMEDDS compositions produced without and with a lipophilic linker, containing 10% nonylphenol (NP), used as polar oil homolog for alkyl phenols with logP >5, were diluted with FeSSIF at a dilution ratio of 30 parts of SMEDDS/70 parts of FeSSIF. An aliquot of 400 μL of the diluted SMEDDS was then placed on the donor compartment of a MatTek Permeation Fixture (EPI-100-FIX). This permeation fixture was used to fasten 8±1 mm diameter, 800±100 μm thickness disks of dermatomed pig ear skin (backside) procured from a local market. The ears were washed and thawed with running water at room temperature before use. The disks were carefully inspected to discard disks with follicular pores or skin defects. The selected disks were placed with the epidermis facing the donor compartment of the permeation fixture. Once the diluted SMEDDS was placed in the donor compartment, the receiver side of the fixture was placed in one of the wells of a 6-well plate and filled with 5 mL of the receiver solution. Care was taken so that no bubbles were trapped between the receiver solution and the disk. The receiver consisted of a phosphate buffer solution with 1.5% of Tween®80 used to simulated lipoproteins in plasma.

The 6-well plate was placed in an incubator shaker with mild agitation at 37° ° C. The receiver solution was sampled after 10, 20, 30, 45, 60 minutes, 2, 3, and 4 hours. At each sampling time, the entire volume of the receiver solution was collected and replaced with a fresh receiver solution. For the analysis of the nonylphenol that permeated into the receiver solution, a 200 μL aliquot of the receiver solution was placed in a 98 well plate for fluorescence intensity measurement (Excitation at 230 nm and emission at 304 nm). The emission signal was compared to a calibration curve (R²=0.9998) produced with standard nonylphenol concentrations in the receiver solution. The nonylphenol concentration in the receiver solution was then used to construct the cumulative permeation curve presented in FIG. 9. The slopes of the linear trend lines in the cumulative permeation curves represent the average flux (F) of drug permeated. The permeability (k) was then calculated as k=F/C_{d} (neglecting the drug concentration in the receiver), where C_{d} is the concentration of the drug in the donor solution. Table 9 presents the composition and permeability of the SMEDDS formulation without and with a lipophilic linker and a nonylphenol solution in ethyl caprate.

As shown by the data in Table 9, the use of SMEDDS(i) produced the largest permeability, being 6.7 times that obtained with nonylphenol in oil. SMEDDS(ii) produced a permeability that was 4.4 times that obtained with nonylphenol in oil. This observation exemplifies the usefulness of the disclosed SMEDDS formulations in improving the transport of polar actives through epithelial tissue.

TABLE 9
Composition, number of phases obtained upon FeSSIF dilution, and nonylphenol
transdermal permeability formulated in (i) lipophilic linker-free, lecithin-
based SMEDDS prepared with the extreme hydrophilic linker Polyaldo ®10-1-
CC; in (ii) lecithin-based SMEDDS prepared with the extreme hydrophilic linker
Polyaldo ®10-1-CC and lipophilic linker Peceol ™; and in (iii)
ethyl caprate (oil) only. Columns (a) through (f) are weight percentage of
(a) lecithin; (b) Peceol ®; (c) Polyaldo ®10-1-CC; (d) FeSSIF;
(e) nonylphenol; (f) ethyl caprate. SMEDDS (i) and (ii) systems correspond
to a dilution line D50, containing 50 parts of surfactant + linkers mixture
for every 50 parts of carrier oil (ethyl caprate), and diluted at 70/30 ratio with FeSSIF

| | | | | | | | | Permeability |
|---|---|---|---|---|---|---|---|---|
| Dilution | %(a) | %(b) | %(c) | % (d) | %(e) | %(f) | Phases | cm/hr |
| 70/30 diluted | 1.35 | 0 | 12.15 | 70 | 3 | 13.5 | 1 | (8 ± 1) · 10⁻⁴ |
| SMEDDS(i) | | | | | | | | |
| 70/30 diluted | 2.025 | 2.025 | 9.45 | 70 | 3 | 13.5 | 1 | (5.3 ± 0.6) · 10⁻⁴ |
| SMEDDS(ii) | | | | | | | | |
| Oil(iii) | 0 | 0 | 0 | 0 | 10 | 90 | 1 | (1.2 ± 0.1) · 10⁻⁴ |

### Example 11. Oral Delivery of Ibuprofen Formulated in SMEDDS with Extreme Hydrophilic Linker (Cc=−6.4)

The SMEDDS composition of Example 5, produced with lipophilic linker Peceol®, extreme hydrophilic linker Caprol® 6GC8, and containing 5% ibuprofen, was used as an oral delivery system with male Sprague-Dawley rats (350±20 g, supplied by Charles River Laboratories Canada). The rats were acclimatized for a week in a temperature-controlled environment with free access to water and food. Rats were randomly assigned to two groups (5 animals in each) depending on whether they received ibuprofen in suspension (control) or in SMEDDS (the composition of Example 5, top row of Table 4). These preparations were administered to animals by oral gavage at a dose of 25 mg/kg. Blood samples (100 μL) were withdrawn through the saphenous vein at 5, 10, 20, 30, 45, 60, 90, 120, 240 and 480 minutes after administration and collected in Heparin-coated tubes. The plasma was separated by centrifugation and stored at −20° C. for analysis. For the control group, ibuprofen was suspended in 0.1% (w/v) of sodium carboxymethyl cellulose (Na-CMC) solution using a high shear homogenizer and hand-shaken once more immediately before use. All the in vivo experiments were conducted according to the guiding principles in the use of animals, as adopted by the University Animal Care Committee (UACC) of the University of Toronto.

To measure the ibuprofen concentration in plasma, 50 μL of plasma samples were diluted with 150 μL acetonitrile, then spiked with 50 μL flufenamic acid solution in acetonitrile as an internal standard. The samples were vortexed, centrifuged, and the supernatant was filtered using a 0.2 μm syringe microfilter. Twenty μL of the filtrate was injected into HPLC. The ratio of ibuprofen AUC to the flufenamic acid AUC was compared against a calibration curve (R²=0.986) to determine the concentration of ibuprofen.

The plasma concentration curves for the SMEDDS formulation and the control are presented in FIG. 10. Table 10 presents the pharmacokinetic parameters after fitting the plasma concentration data to a single compartment, first-order model. The value of tₘₐₓ is the time when the plasma concentration reaches its peak (Cₘₐₓ.). AUC₀₋₈ₕ is the area under the plasma concentration curve, from the time of dosing until 8 hours after dosing. The value of kₐ is the first order adsorption constant, and k10 is the first-order elimination constant. The data in Table 10 show that, compared to the control, the SMEDDS formulation produced a significant increase in Cₘₐₓ, AUC₀₋₈ₕ, and kₐ was obtained (p<0.05). The SMEDDS produced an increase in AUC (proportional to drug uptake) of 3.9 times compared to the control and an increase of 3.5 times in Cₘₐₓ.

TABLE 10
Pharmacokinetic parameters for orally administered ibuprofen
using the SMEDDS formulation of Example 5 containing 5%
ibuprofen and an aqueous suspension of 5% ibuprofen in
0.1% (w/v) of sodium carboxymethylcellulose solution.

| Parameter | Unit | SMEDDS | Control |
|---|---|---|---|
| tₘₐₓ | min | 22.1 ± 8.0 | 42 ± 22 |
| Cₘₐₓ | μg/ml | 28.9 ± 3.4 | 8.2 ± 1.0 |
| AUC_{0-8 h} | μg/ml*min | (7.3 ± 2.2)*10³ | (1.85 ± 0.79)*10³ |
| kₐ* | 1/min | 0.23 ± 0.11 | 0.08 ± 0.04 |
| k₁₀ | 1/min | 0.004 ± 0.002 | 0.012 ± 0.012 |

### Example 12. Clove Oil (Eugenol) in SMEDDS with Extreme Hydrophilic Linker (Cc=−7.4) and Diluted with Deionized (DI) Water

A fully-dilutable SMEDDS was produced by mixing 10 parts (by mass) of lecithin with 90 parts of the hydrophilic linker Polyaldo®10-1-CC using a vortex-mixer. A prescribed ratio of 35 parts (by mass) of ethyl caprate and 65 parts of the lecithin+hydrophilic linker mixture was then mixed using a vortex-mixer. 95 parts (by mass) of the resulting mixture were then mixed with 5 parts of clove oil (70% eugenol, Cc=+2.8) used as model polar oil. The resulting solution was diluted with deionized water. The diluted systems were vortex-mixed and then left to equilibrate for two weeks at room temperature. Table 11 summarizes the phase behavior obtained upon dilution with DI water.

TABLE 11
Composition, number of phases obtained upon water dilution, viscosity and
drop size of lipophilic linker-free, lecithin-based SMEDDS prepared with
the extreme mhydrophilic linker Polyaldo ®10-1-CC, 5% clove oil (eugenol)
and diluted with deionized (DI) water. Columns (a) through (f) are weight
percentage of (a) lecithin; (b) lipophilic linker; (c) Polyaldo ®10-1-CC
(d) DI water; (e) clove oil (eugenol); (f) ethyl caprate. The system corresponds
to a dilution line D65, containing 65 parts of surfactant + hydrophilic
linker mixture for every 35 parts of carrier oil (ethyl caprate).

| | | | | | | | | Viscosity | Size |
|---|---|---|---|---|---|---|---|---|---|
| Dilution | %(a) | %(b) | %(c) | % (d) | %(e) | %(f) | Phases | cP | nm |
| SMEDDS | 6.2 | 0.0 | 55.6 | 0 | 5 | 33.2 | 1 | | N.D |
| 10/90 | 5.6 | 0.0 | 50.0 | 10 | 4.5 | 29.9 | 1 | 650 ± 50 | 53 ± 5 |
| 20/80 | 4.9 | 0.0 | 44.5 | 20 | 4 | 26.6 | 1 | 170 ± 20 | 48 ± 5 |
| 30/70 | 4.3 | 0.0 | 38.9 | 30 | 3.5 | 23.3 | 1 | 130 ± 20 | 30 ± 4 |
| 40/60 | 3.7 | 0.0 | 33.3 | 40 | 3 | 20.0 | 1 | 120 ± 15 | 2 ± 1 |
| 50/50 | 3.1 | 0.0 | 27.8 | 50 | 2.5 | 16.6 | 1 | 90 ± 10 | 2 ± 1 |
| 60/40 | 2.5 | 0.0 | 22.2 | 60 | 2 | 13.3 | 1 | 21 ± 5 | 2 ± 1 |
| 70/30 | 1.9 | 0.0 | 16.7 | 70 | 1.5 | 10.0 | 1 | 7 ± 2 | 3 ± 1 |
| 80/20 | 1.2 | 0.0 | 11.1 | 80 | 1 | 6.7 | 1 | 2 ± 2 | 25 ± 3 |
| 90/10 | 0.6 | 0.0 | 5.6 | 90 | 0.5 | 3.3 | 1 | 1 ± 1 | 42 ± 3 |
| 99/1 | 0.1 | 0.0 | 0.6 | 99 | 0.05 | 0.3 | 1 | 1 ± 1 | 28 ± 3 |

The data in Table 11 confirms the fully-dilutable nature of the D65 composition. The table further confirms that the polar active, eugenol, in clove oil can also have solubilities close to 1 wt % and log P values closer to 1. The example also demonstrates the ability to dilute the disclosed SMEDDS with pure water.

### Example 13. Nonylphenol in HSA-Gelled SMEDDS with an Extreme Hydrophilic Linker

The SMEDDS was produced by first mixing 10 parts (by mass) of lecithin with 90 parts of the hydrophilic linker Polyaldo®10-1-CC (Cc=−7.4) using a vortex-mixer. A prescribed ratio of 40 parts (by mass) of limonene (racemic mixture, technical grade) and 60 parts of the Lecithin+hydrophilic linker mixture was then mixed using a vortex-mixer. 95 parts (by mass) of the resulting mixture were then mixed with 5 parts of nonylphenol used as model polar oil. The resulting solution was used as the organic solvent for the low molecular weight organogelator 12-hydroxystearic acid (12-HSA). The organogelator was added at 10 wt. % in mixture with the SMEDDS. The mixture was heated in a temperature-controlled water bath to 80° C. and then maintained at that temperature until the gelator was fully dissolved in the oil phase, producing a transparent/translucent solution. After vortex mixing, the samples were cooled down to room temperature, where the system solidified for 48 hours.

The rheological behavior of the resulting gel was evaluated using a Carri-Med CSL2 Rheometer (TA Instruments, USA). A 4-cm stainless steel parallel-plate geometry was attached, and a newly prepared hot melted gel was poured onto the lower rheometer plate. The lower plate temperature was controlled via Peltier Plate, initially set at 80° C., then cooled down to 20° C. in 90 minutes, and then left to rest at 20ºC for 90 minutes. At that point, the oscillatory experiment was commenced, and the sample was heated from 20° C. to 80° C. at the rate of 0.8° C./min. The oscillatory test was conducted using a gap size of 200 μm, maintaining the shear stress (τ), shear strain (γ) and frequency constant at 75 Pa, 0.001 (0.1%) and 10 rad/s, respectively. The dynamic moduli G′ and G″ (Pa) were recorded during the heating cycle as a function of temperature. FIG. 11 presents these values of elastic (G′) and shear (G″) moduli for the gelled D60 SMEDDS as a function of temperature. Pure gel behavior (G′>G″) was observed when the temperature was lower than 30° C. This example illustrates that, contrary to previous observations in the literature, it is possible to produce gels with low molecular weight gelators and oils in the presence of a high concentration of surfactants.

The SMEDDS formulation (Lec: HL 10:90, D60, 5% nonylphenol, 10% 12-HSA) was used to prepare the drug-loaded gelled SMEDDS. 32±5 mg of melted gel SMEDDS was poured into aluminum pans (6 mm diameter, 2 mm height) and let to cool down and solidify at room temperature for 24 hrs. The disk-shaped gels were then placed in 1-dram glass vials, and 3 mL of FeSSIF was added. The vials were placed into an isothermal shaker set at 100 rpm and 25° C. At specific time intervals, the aqueous phase of the vials was removed for analysis and the vials were re-filled with fresh FESSIF. At each sampling time, the entire volume of the receiver solution was collected and replaced with a fresh receiver solution. For the analysis of the nonylphenol that permeated into the receiver solution, a 200 μL aliquot of the receiver solution was placed in a 98 well plate for fluorescence intensity measurement (Excitation at 230 nm and emission at 304 nm). The emission signal was compared to a calibration curve (R²=0.9998) produced with standard nonylphenol concentrations in the receiver solution. The concentration of nonylphenol in the receiver solution was then used to construct the cumulative release versus the square root of time, as shown in FIG. 12. The linear trendline in FIG. 12 is typical of controlled release systems that regulate the release of the active via diffusion. The release time can be estimated as (1/slope of trendline)\hat{ }2, which is 27 hours for the system in FIG. 12. In the absence of the gelling agent, the release is nearly instantaneous, on the scale of seconds to minutes.

### Example 14. Nonylphenol in Phytosterol-Gelled SMEDDS with Extreme Hydrophilic Linker

The SMEDDS was produced by first mixing 10 parts (by mass) of lecithin with 90 parts of the hydrophilic linker Polyaldo® 10-1-CC (Cc=−7.4) using a vortex-mixer. A prescribed ratio of 40 parts (by mass) of limonene (racemic mixture, technical grade) and 60 parts of the Lecithin+hydrophilic linker mixture was then mixed using a vortex-mixer. 95 parts (by mass) of the resulting mixture were then mixed with 5 parts of nonylphenol used as model polar oil. The resulting solution was used as the organic solvent for the low molecular weight with a mixture of 18 and 20 wt % of organogelators β-sitosterol and γ-oryzanol mixed at a weight ratio of 1:1. The mixture was heated in a temperature-controlled water bath to 90° C. and then maintained at that temperature until the gelator was fully dissolved in the oil phase, producing a transparent/translucent solution. After vortex mixing, the samples were cooled down to room temperature, where the system solidified for 48 hours.

The rheological behavior of the resulting gel was evaluated using a Carri-Med CSL2 Rheometer (TA Instruments, USA) following the methodology indicated in Example 13. FIG. 13 presents these values of elastic (G′) and shear (G″) moduli for the gelled D60 SMEDDS as a function of temperature. Pure gel behavior (G′>G″) was observed when the temperature was lower than 42° C. for the system with 20 wt % gelators and lower than 28° C. for the system with 18 wt % gelators. This example further illustrates the production of gelled SMEDDS and that the mechanical properties of the gel such as G′, G″, the melting temperature can be adjusted using different gelators and their concentration.

32±5 mg of melted gelled SMEDDS formulation (Lec: HL 10:90, D60, 5% nonylphenol, and 18 and 20 wt % organogelator mixture) was poured into aluminum pans and let to cool down and solidify at room temperature for 24 hrs. The disk-shaped gels were then placed in 1-dram glass vials, and 3 mL of FeSSIF was added. The vials were placed into an isothermal shaker set at 100 rpm and 25° C. At specific time intervals, the aqueous phase of the vials was removed for analysis and the vials were re-filled with fresh FESSIF. At each sampling time, the entire volume of the receiver solution was collected and replaced with a fresh receiver solution. For the analysis of the nonylphenol that permeated into the receiver solution, a 200 μL aliquot of the receiver solution was placed in a 98 well plate for fluorescence intensity measurement. The nonylphenol concentration in the receiver solution was then used to construct the cumulative release versus the square root of time for the 18 wt % and 20 wt % gelator systems, as shown in FIG. 14. The experimental data for both systems were fitted with linear trendlines typical of diffusion-controlled release. The release time, estimated as (1/slope)\hat{ }2, is 285 hours (12 days) for 18 wt % gelators and 641 hours (27 days) for 20 wt % gelators.

### Example 15. Encapsulated SMEDDS with Extreme Hydrophilic Linker

A D55 SMEDDS was produced by first mixing 10 parts (by mass) of lecithin with 90 parts of the hydrophilic linker Polyaldo® 10-1-CC (Cc=−7.4) using a vortex-mixer. A prescribed ratio of 45 parts (by mass) of limonene (racemic mixture, technical grade) and 55 parts of the Lecithin+hydrophilic linker mixture was then mixed using a vortex-mixer. This D55 SMEDDS was then loaded with 5 wt % nonylphenol used as model polar oil. This loaded SMEDDS composition was then encapsulated with three coating agents, EUDRAGUARD® natural non-enteric coating agent; EUDRAGIT® FL 30 D-55; and PROTECT™ ENTERIC.

To encapsulate the D55 SMEDDS with EUDRAGUARD® (acetylated Starch E1420, Evonik), 7.5 g of this polymer dissolved in 100 ml of distilled water+5 g of D60 SMEDDS, for a final loading of 40% loading in the final solid. 12.5% total core+coat concentration. The mixture was stirred for 1 hr before use. Stirring continued during spray drying.

To encapsulate the D55 SMEDDS with EUDRAGIT L30 D-55 (30% dispersion of methacrylic acid and ethyl acrylate copolymer NF, Evonik), 25 mL of the polymer suspension (equal to 7.5 g solid materials) were dissolved in 33 ml of distilled water+5 g of SMEDDS, for a final loading of 40% SMEDDS in the final solid. The mixture was stirred for 1 hr before use. Stirring continued during spray drying.

To encapsulate the D55 SMEDDS with PROTECT™ ENTERIC (shellac+sodium alginate, Sensient® Pharmaceuticals), 2.5 g of Protect Clear SA powder (Na-alginate), dissolved in 100 ml of distilled water, stir 30 min and then add 18.25 mL of polymer suspension, Protect ENLA (equal to 5.25 g solid materials)+5 g of SMEDDS, for a final loading of 40% (6:4, polymer:oil). The mixture stirred for 1 hr before use; stirring the feed continues during spray drying

The suspensions of the D55 SMEDDS and each coating agent were then spray-dried using a Model HT-RY 1500 spray dryer (Zhengxhou Hento Michinery Co. Ltd) equipped with a Imm nozzle, operating with an air pressure of 25 psi, an inlet temperature of 70° C., and a flow rate of 6 mL/min. The resulting powders were then subjected to release tests in acidic conditions to simulate gastric conditions and near-neutral pH to simulate intestinal conditions.

For the release test, 50 mg of the finished product powders (containing 20 mg of SMEDDS) were placed in four 15-mL falcon centrifuge tubes. 5 mL of an aqueous HCl solution at pH 1.3 was added to the samples. The tubes were shaken for 1 hr in a temperature-controlled shaker (37° C., 100 rpm). After one hour, the samples were centrifuged at 2000 rpm for 5 min. The supernatants were removed for analysis. The solids left at the bottom of the test tube were then mixed with 5 ml of FeSSIF, and the tubes were shaken for 1 hr at 37° C. and 100 rpm. The aqueous HCl solution at pH 1.3 and FeSSIF were used as references for the release in gastric and intestinal conditions. The supernatants were removed for analysis. The concentration of the released nonylphenol in the supernatant was determined via fluorescence spectroscopy, using the same method employed in Example 10.

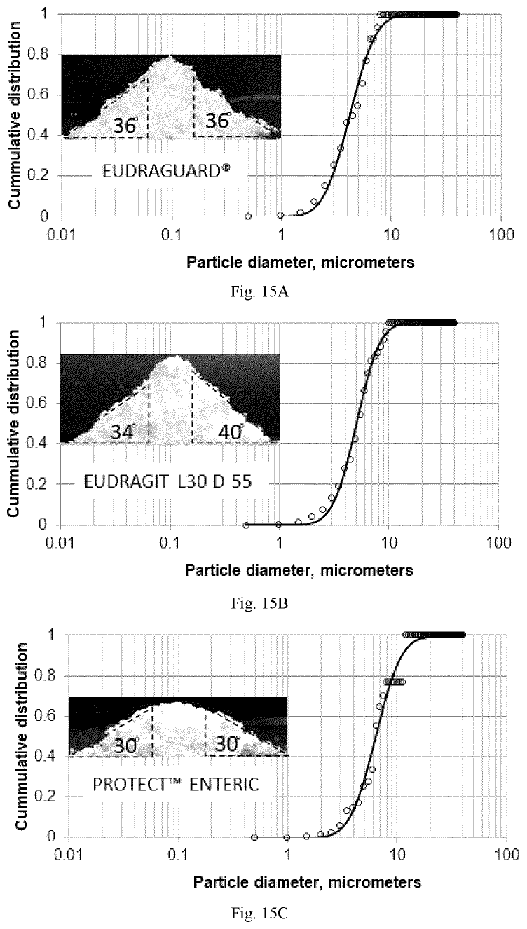

The particle size distribution obtained with each of the encapsulated SMEDDS was determined via optical microscopy, observing samples of the particles placed on a glass slide with an Olympus BX-51 microscope used in transmitted light mode. The micrographs obtained using a 50× objective were then analyzed using the ImageJ software's particle analysis tool. Volume-based cumulative size distribution for EUDRAGUARD®, EUDRAGIT® FL 30 D-55, and PROTECT™ ENTERIC are shown in FIGS. 15.A, 15.B, and 15.C, respectively. The inset in each Figure shows the angle of repose images obtained using the hollow cylinder test method [35].

TABLE 12
Composition, % release in HCl, % release in FeSSIF, average particle size and angle of repose
of encapsulated D55 SMEDDS formulated with lecithin, extreme hydrophilic linker Polyaldo ®10-1-CC
and limonene with nonylphenol as model polar oil. Columns (a) through (e) are weight percentage
of (a) lecithin; (b) Polyaldo ®10-1-CC; (c) nonylphenol; (d) limonene, (e) encapsulating
polymer. Column (f) presents the % release of the SMEDDS in HCl solution, column (g) presents
the % release of the SMEDDS in FeSSIF, column (h) presents the average particle size of the
encapsulated SMEDDS; and (i) is the angle of repose of the encapsulated SMEDDS.

| Encapsulating | | | | | | | | | |
|---|---|---|---|---|---|---|---|---|---|
| polymer | %(a) | %(b) | %(c) | %(d) | %(e) | (f)% | (g)% | (h)μm | (i)deg. |
| EUDRAGUARD ® | 2.66 | 23.94 | 2.00 | 11.40 | 60.00 | 13 ± 1% | 100 ± 1% | 4.2 ± 1.5 | 36 ± 1 |
| EUDRAGIT ® | 2.66 | 23.94 | 2.00 | 11.40 | 60.00 | 17 ± 1% | 91 ± 1% | 5.1 ± 1.5 | 37 ± 3 |
| PROTECT ™ | 2.66 | 23.94 | 2.00 | 11.40 | 60.00 | 0 ± 1% | 53 ± 1% | 6.4 ± 1.5 | 30 ± 1 |

### Example 16. Oral Delivery of CBD Formulated in SMEDDS and Encapsulated SMEDDS with Extreme Hydrophilic Linker (Cc=−7.4)

The control CBD composition was prepared by adding 1 g of CBD in 99 g of medium chain triglyceride (MCT) oil (Organic Pure C8 MCT Oil, 99.2% C8 triglycerides), for a final CBD concentration of 9.6 mg/mL.

A D70 SMEDDS was produced by first mixing 10 parts (by mass) of lecithin with 45 parts of the hydrophilic linker Polyaldo®10-1-CC (Cc=−7.4) and 45 parts of the hydrophilic linker Dermofeel® G6CY (Cc=−3) using a vortex-mixer. Considering the molecular weights for these hydrophilic linkers, shown in Table 15, this 1:1 mass ratio represents a molar ratio of 1.13 moles of Polyaldo®10-1-CC (Cc=−7.4) to 1.695 mol parts of Dermofeel® G6CY (Cc=−3). Using the molar fraction linear mixing rule for Cc used by Zarate et al. [22], then the Cc for the mixture is (−7.4)*1.13/(1.13+1.695)+(−3)*1.695/(1.13+1.695)=−4.76, which is within the −5+20% boundary set for the least negative value of the Cc of the combined extreme hydrophilic linker. A prescribed ratio of 15 parts (by mass) of limonene (racemic mixture, technical grade), 15 parts of ethyl oleate (for a total of 30 parts of oil) were added to 70 parts of the Lecithin+hydrophilic linker mixture (i.e., the mixture of 45 parts Polyaldo®10-1-CC and 45 parts of Dermofeel® G6CY) and then mixed using a vortex-mixer. 80 parts of this D70 SMEDDS where then vortex-mixed with 20 parts of CBD to produce a 20 wt % loaded D70) SMEDDS. This will be referred to as the 20% CBD-D70 SMEDDS composition.

To encapsulate the 20% CBD-D70 SMEDDS with EUDRAGIT L30 D-55 (30% dispersion of methacrylic acid and ethyl acrylate copolymer NF, Evonik), 164.4 mL of the polymer suspension (equal to 49.3 g solid materials) were dissolved in a mixture of 33 mL of FeSSIF and 134 g of distilled water. Once this suspension was homogenized, 33 g of 20% CBD-D70 SMEDDS were added, for a final loading of 40 wt % of 20% CBD-D70) SMEDDS in the final solid. The mixture was stirred for 1 hr before drying. Stirring continued during spray drying, a process that was undertaken using a Model HT-RY 1500 spray dryer (Zhengxhou Hento Michinery Co. Ltd) equipped with a 1 mm nozzle, operating with an air pressure of 25 psi, an inlet temperature of 60° C., and a flow rate of 6 mL/min. To confirm the CBD loading in the dry powder, a solvent extraction procedure was undertaken, followed by HPLC determination of the CBD concentration. The resulting concentration was determined to be 7.1% CBD in the encapsulated 20% CBD-D70 SMEDDS, indicating that the encapsulation efficiency was 89%.

To conduct the pharmacokinetic studies with these three compositions (control CBD, 20% CBD-D70 SMEDDS, and encapsulated 20% CBD-D70 SMEDDS), male Sprague-Dawley rats (250±20 g, supplied by Envigo, Indianapolis, In, USA) were used as animal models. The pharmacokinetic study was carried out by Nucro-Technics (Scarborough, ON, Canada), a contracted facility authorized to conduct studies with cannabinoids and approved to conduct animal studies using animal care protocols that meet ethical practices for animal studies in Canada. The rats were acclimatized for a week in a temperature-controlled environment with free access to water and food. Rats were randomly assigned to three groups, (a) 10 rats in a control group with CBD dissolved in medium chain triglyceride (MCT), (b) 12 rats in a SMEDDS group with CBD dissolved in a liquid SMEDDS formulation, and finally (c) 8 rats in a group does with CBD formulated in powder encapsulated SMEDDS. Table 13 presents the summary of the dosing conditions for these three test groups. Each test group was subdivided into two (for CBD control and CBD Powder) or three (for CBD SMEDDS) sub-groups to ensure that the number of blood sampling events was 6 or less for each rat. A total of 11 sampling events considered at 10 min, 20 min, 30 min, 45 min, 1 h, 1.5 h, 2 h, 4 h, 6 h, 8 h and 10 h. At each sampling event, blood samples (450±50 μL) were withdrawn from the jugular vein (or the orbital sinus) into tubes containing anticoagulant K₂EDTA. Following the collection of blood samples, the blood was placed in a refrigerated centrifuge for 15 minutes to separate the plasma, and the recovered plasma was stored in cryovials frozen at −60° C. The plasma samples were analyzed using a LC-MS/MS method for plasma quantitation of CBD and 7-COOH-CBD, having a limit of quantification of 5.0 ng/mL. The LC-MS/MS method involved the use of a mobile phase A: 70% Methanol, 5 mM Ammonium Acetate, 0.1% Formic Acid; and mobile phase B: 90% Methanol, 5 mM Ammonium Acetate, 0.1% Formic Acid. The flow rate was 0.5 mL/min and the gradient conditions were as follows 0-3 min, 80% A and 20% B: 3.01-6 min, 100% B: 6.01-8 min, 80% A. An ACE Excel 5 Super C18 (75×3.0 mm, 5 μm) chromatography column was used. Temperature of column: 25° C. Spectrometer mass conditions: Gas Temperature: 350° C. Capillary: 4 KV. Gas Flow: 13 L/min

TABLE 13
Summary of test groups used in the pharmacokinetic study of CBD, including
dose, dose concentration, dose volume and dosing instructions.

| Research group | CBD control | CBD SMEDDS | CBD Powder |
|---|---|---|---|
| Dose | 10 | 10 | 10 |
| (mg/Kg) | | | |
| CBD Dosing conc. | 9.6 | 1.28 | 1.061 |
| (mg/mL) | | | |
| Dosing volume | 1.04 | 7.81 | 10.37 |
| (ml/Kg) | | | |
| Formulation | | | |
| Dosing method | The required | 0.268 g of | 40 mL phosphoric |
| | dosing volume | SMEDDS (20% | acid solution (pH3) + |
| | was administered | CBD) diluted with | 0.6 g powder |
| | by oral gavage | 38.95 mL FeSSIF, | SMEDDS to produce |
| | | administered by | a suspension dosed |
| | | oral gavage. | by oral gavage |

The plasma concentration curves for the CBD control, the 20% CBD-D70 SMEDDS (referred to as SMEDDS in FIG. 16), and the encapsulated 20% CBD-D70 SMEDDS (referred to as powder in FIG. 16) are presented in FIG. 16. Table 14 presents the pharmacokinetic parameters after fitting the plasma concentration data to a noncompartmental analysis for extravascular systems programmed in PKSolver [36]. The reason that a non-compartmental model had to be used is because of the double-peak feature of the SMEDDS and the powder curves in FIG. 16, which cannot be reproduced by a typical single compartment model. The value of tₘₐₓ is the time when the plasma concentration reaches its peak (Cₘₐₓ.). AUC₀₋₁₀ₕ is the area under the plasma concentration curve, from the time of dosing until 10 hours after dosing. The value of AUC_{0-inf} represents an estimation of the area under the curve extrapolated to an infinite release time, estimated based the decay trend obtained with the last 4 points of the curve.

TABLE 14
Pharmacokinetic parameters for orally administered CBD
in the control, in the liquid (SMEDDS) 20% CBD-D70 SMEDDS,
and in the encapsulated (powder) 20% CBD-D70 SMEDDS.

| Parameter | Unit | Control | SMEDDS | Powder |
|---|---|---|---|---|
| tₘₐₓ | hours | 1.5 | 0.5 | 0.33 |
| Cₘₐₓ | ng/ml | 78 | 121 | 162 |
| AUC_{0-10 h} | ng/ml*hr | 404 | 454 | 520 |
| AUC_{0-inf} | ng/ml*hr | 423 | 840 | 578 |

As illustrated by the tₘₐₓ values in Table 14, the SMEDDS (20% CBD-D70 SMEDDS) and powder (encapsulated 20% CBD-D70 SMEDDS) compositions reduce the time to reach Cₘₐₓ by at least 65% of the time required by the control. This is definitely an advantageous feature of these formulas as it facilitates the potential for fast-acting effects of the cannabinoid. The Cₘₐₓ obtained with the SMEDDS is more than 50% greater than the Cₘₐₓ of the control, and the Cₘₐₓ obtained with the powder more than doubled the Cₘₐₓ of the control. The 10-hour area under the curves (AUC 0-10 h) were about 10% and 30% larger for SMEDDS and the powder, respectively, as compared to the control. The assessed infinite absorption (AUC_{0-inf}) was substantially larger for the SMEDDS (nearly twice that of the control) because the plasma concentration of CBD was nearly constant in the last four measurements for the SMEDDS curve.

While Example 3 shows that the use of conventional hydrophilic linker Dermofeel® could not produce a fully dilutable formulation, Example 16 shows that a conventional hydrophilic linker when used in combination with an extreme hydrophilic linker like Polyaldo®10-1-CC, can result in a fully dilutable system when the combination/mixture has a Cc of about −5 or more negative than about −5.

TABLE 15
Characteristic curvatures of selected biobased surfactants.

| | | MW | vs/as | L |
|---|---|---|---|---|
| Surfactant | Cc | (g/mol) | (Å) | (Å) |
| Oleic acid | 0 ± 0.1 | 282 | § | § |
| Sodium oleate | −1.7 ± 0.1 | 304 | 8.4 | 30 |
| Sodium octanoate | −2.1 ± 0.1 | 166 | 4.6 | 12 |
| Soybean lecithin | +5.5 ± 1.3 | 750 | 11.7 | 34 |
| Glycerol monooleate | +6.6 ± 1.5 | 450 | § | § |
| Polyglycerol-6-caprylate | −3.0 ± 0.7 | 590 | 9.9 | 19 |
| (Dermofeel ® G6CY) | | | | |
| Polyglycerol-6-caprylate | −6.4 ± 1.28 | 593 | | |
| (Caprol ® 6GC8) | | | | |
| Sorbitan monolaurate, Span 20 | +3.5 ± 0.8 | 346 | § | § |
| Tween 20 | −4.4 ± 1.0 | 1228 | 24.3 | 23 |
| Tween 80 | −3.0 ± 0.7 | 1310 | 25.8 | 34 |
| Sodium lauroyl sarcosinate | −4.2 ± 0.2 | 293 | 5.8 | 20 |
| Sucrose palmitate | −0.8 ± 0.2 | 581 | 17.8 | 23 |
| Sucrose distearate | +4 ± 0.2 | 875 | 12.0 | 34 |
| Disodium stearoyl capryl | −5 ± 0.2 | 457 | 7.9 | 34 |
| glutamate | | | | |
| C18 sophorolipid | +7 ± 1.5 | 688 | 13.0 | 34 |
| Monorhamnolipid | −1.4 ± 0.3 | 577 | 14.5 | 16 |
| Dirhamnolipid | −1.3 ± 0.3 | 650 | 14.2 | 16 |
| GY19 lipopeptides | +4.9 ± 1 | 1050 | NA | NA |
| Polyglyceryl-10-caprylate | −7.4 ± 1 | 885 | NA | NA |
| (Polyaldo ® 10-1-CC) | | | | |
| C12-C14 polyglycoside | +0.5 ± 0.2 | 480 | 16.1 | 25 |
| Sorbitan monooleate (Span 80) | +5.0 ± 1 | 430 | § | § |
| C6 polyglucoside | −2.2 ± 0.5 | 380 | 8.8 | 12 |

Note:
lipophilic linkers are noted with the symbol “§.”

### REFERENCES CITED

[1] E. J. Acosta, M. Nouraei, Delivery Systems: Improving Food Quality, Safety and Potential Health Benefits, in: O. Ramos, R. Pereira, M. Cerqueira, J. Teixeira, A. Vicente (Eds.), Adv. Process. Technol. Bio-Based Nanosyst. Food, first, CRC Press, Boca Raton, Florida, 2020: pp. 153-188.
[2] G. Cevc, Lipid vesicles and other colloids as drug carriers on the skin, Adv. Drug Deliv. Rev. 56 (2004) 675-711. https://doi.org/10.1016/j.addr.2003.10.028.
[3] J. R. Pappenheimer, K. Z. Reiss, Contribution of solvent drag through intercellular junctions to absorption of nutrients by the small intestine of the rat, J. Membr. Biol. 100 (1987) 123-136. https://doi.org/10.1007/BF02209145.
[4] K. M. Hämäläinen, K. Kananen, S. Auriola, K. Kontturi, A. Urtti, Characterization of paracellular and aqueous penetration routes in cornea, conjunctiva, and sclera, Investig. Ophthalmol. Vis. Sci. 38 (1997) 627-634.
[5] E. J. Acosta, Bioavailability of nanoparticles in nutrient and nutraceutical delivery, Curr. Opin. Colloid Interface Sci. 14 (2009) 3-15. https://doi.org/10.1016/j.cocis.2008.01.002.
[6] O. Lieleg, I. Vladescu, K. Ribbeck, Characterization of particle translocation through mucin hydrogels. Biophys. J. 98 (2010) 1782-1789. https://doi.org/10.1016/j.bpj.2010.01.012.
[7] J. Chu, Y. L. Cheng, A. V. Rao, M. Nouraei, S. Zarate-Muñoz, E. J. Acosta, Lecithin-linker formulations for self-emulsifying delivery of nutraceuticals, Int. J. Pharm. 471 (2014) 92-102. https://doi.org/10.1016/j.ijpharm.2014.05.001.
[8] C. Fruijtier-Polloth, Safety assessment on polyethylene glycols (PEGs) and their derivatives as used in cosmetic products, Toxicology. 214 (2005) 1-38. https://doi.org/10.1016/j.tox.2005.06.001.
[9] F. J. Schweigert, A. Trüpschuch, C. Hantschel, Modulation of absorption of beta-carotene and tissue accumulation of beta-carotene and vitamin a by different surfactants in rats, Ann. Nutr. Metab. 46 (2002) 200-204. https://doi.org/10.1159/000065407.

[10] M. Mohamed, A. S. Abu Lila, T. Shimizu, E. Alaaeldin, A. Hussein, H. A. Sarhan, J. Szebeni, T. Ishida, PEGylated liposomes: immunological responses, Sci. Technol. Adv. Mater. 20) (2019) 710-724. https://doi.org/10.1080/14686996.2019.1627174.
[11] D. A. Cobb, N. A. Smith, B. J. Edagwa, J. E. M. McMillan, Long-acting approaches for delivery of antiretroviral drugs for prevention and treatment of HIV: a review of recent research, Expert Opin. Drug Deliv. 17 (2020) 1-12. https://doi.org/10.1080/17425247.2020.1783233.
[12] D. G. Leach, S. Young, J. D. Hartgerink, Advances in immunotherapy delivery from implantable and injectable biomaterials, Acta Biomater. 88 (2019) 15-31. https://doi.org/10.1016/j.actbio.2019.02.016.
[13] X. Y. Xuan, Y. L. Cheng. E. Acosta, Lecithin-linker microemulsion gelatin gels for extended drug delivery, Pharmaceutics. 4 (2012) 104-129. https://doi.org/10.3390/pharmaceutics4010104.
[14] S. Z. Munoz, R. Zhadan, E. Acosta, Design of nonionic micelle-laden polysaccharide hydrogels for controlled delivery of hydrophobic drugs, Int. J. Pharm. 526 (2017) 455-465. https://doi.org/10.1016/j.ijpharm.2017.04.062.
[15] Y. S. R. Elnaggar, W. M. El-Refaie, M. A. El-Massik, O. Y. Abdallah, Lecithin-based nanostructured gels for skin delivery: An update on state of art and recent applications, J. Control. Release. 180 (2014) 10-24. https://doi.org/10.1016/j.jconrel.2014.02.004.
[16] Z. Ćetković, S. Cvijić, D. Vasiljević, Formulation and characterization of novel lipid-based drug delivery systems containing polymethacrylate polymers as solid carriers for sustained release of simvastatin, J. Drug Deliv. Sci. Technol. 53 (2019). https://doi.org/10.1016/j.jddst.2019.101222.
[17] K. Iwanaga, T. Sumizawa, M. Miyazaki, M. Kakemi, Characterization of organogel as a novel oral controlled release formulation for lipophilic compounds, Int. J. Pharm. 388 (2010) 123-128. https://doi.org/10.1016/j.ijpharm.2009.12.045.
[18] O. Chung, Sterol-Based Organogel Drug Delivery Systems, University of Toronto, 2012. http://hdl.handle.net/1807/42386.
[19] C. Sun, Y. Gui, R. Hu, J. Chen, B. Wang, Y. Guo, W. Lu, X. Nie, Q. Shen, S. Gao, W. Fang, Preparation and Pharmacokinetics Evaluation of Solid Self-Microemulsifying Drug Delivery System (S-SMEDDS) of Osthole, AAPS PharmSciTech. 19 (2018) 2301-2310. https://doi.org/10.1208/s12249-018-1067-3.
[20] T. Yi, J. Wan, H. Xu, X. Yang, A new solid self-microemulsifying formulation prepared by spray-drying to improve the oral bioavailability of poorly water soluble drugs, Eur. J. Pharm. Biopharm. 70 (2008) 439-444. https://doi.org/10.1016/j.ejpb.2008.05.001.
[21] M. Nouraei, E. J. Acosta, Predicting solubilisation features of ternary phase diagrams of fully dilutable lecithin linker microemulsions, J. Colloid Interface Sci. 495 (2017) 178-190. https://doi.org/10.1016/j.jcis.2017.01.114.
[22] S. Zarate-Muñoz, F. Texeira De Vasconcelos, K. Myint-Myat, J. Minchom, E. J. Acosta, A Simplified Methodology to Measure the Characteristic Curvature (Cc) of Alkyl Ethoxylate Nonionic Surfactants, J. Surfactants Deterg. 19 (2016) 249-263. https://doi.org/10.1007/s11743-016-1787-x.
[23] A. Ghayour, E. Acosta, Characterizing the Oil-like and Surfactant-like Behavior of Polar Oils, Langmuir. 35 (2019) 15038-15050. https://doi.org/10.1021/acs.langmuir.9b02732.
[24] S. Abbott, Practical Surfactants, (2019).
[25] A. Mouri, O. Diat, A. El Ghzaoui, I. Ly, C. Dorandeu, J. C. Maurel, J. M. Devoisselle, P. Legrand, Development of pharmaceutical clear gel based on Peceol®, lecithin, ethanol and water: Physicochemical characterization and stability study, J. Colloid Interface Sci. 457 (2015) 152-161. https://doi.org/10.1016/j.jcis.2015.06.010.
[26] S. Sundar, M. Nouraei, T. Latta, E. Acosta, Hydrophilic-lipophilic-difference (HLD) guided formulation of oil spill dispersants with biobased surfactants, Tenside, Surfactants, Deterg. 56 (2019). https://doi.org/10.3139/113.110643.
[27] E. Acosta, Regulatory aspects of nutrient delivery systems, first, Woodhead Publishing, Boca Raton, Florida, 2008. https://doi.org/10.1533/9781845694210.4.429.
[28] E. J. Acosta, M. A. Le, J. H. Harwell, D. A. Sabatini, Coalescence and solubilization kinetics in linker-modified microemulsions and related systems, Langmuir. 19 (2003) 566-574. https://doi.org/10.1021/la0261693. [29] E. J. Acosta, J. S. Yuan, A. S. Bhakta, The characteristic curvature of ionic surfactants, J. Surfactants Deterg. 11 (2008) 145-158. https://doi.org/10.1007/s11743-008-1065-7. [30] E. J. Acosta, S. Sundar, How to Formulate Biobased Surfactants Through the HLD-NAC Model, in: D. Hayes, D. Solaiman, R. Ashby (Eds.), Biobased Surfactants, 2nd ed., AOCS Press, London, U. K., 2019: pp. 471-510. https://doi.org/10.1016/B978-O-12-812705-6.00015-0.
[31] A. Ghayour, E. J. Acosta, Erratum: Characterizing the Oil-like and Surfactant-like Behavior of Polar Oils, Langmuir. 36 (2020) 3276-32771. https://doi.org/10.1021/acs.langmuir.0c00510.
[32] R. Haag, J. F. Stumbé, A. Sunder, H. Frey, A. Hebel, An approach to core-shell-type architectures in hyperbranched polyglycerols by selective chemical differentiation, Macromolecules. 33 (2000) 8158-8166. https://doi.org/10.1021/ma000831p.
[33] V. Tantishaiyakul, N. Worakul, W. Wongpoowarak, Prediction of solubility parameters using partial least square regression, Int. J. Pharm. 325 (2006) 8-14. https://doi.org/10.1016/j.ijpharm.2006.06.009.
[34] S. Sun, L. Li, J. Wang, S. Song, P. He, Z. Fang, Reconstruction and measurement of irregular karst caves using BLST along the shield metro line, Appl. Sci. 10 (2020). https://doi.org/10.3390/app10010392.
[35] H. M. Beakawi Al-Hashemi, O. S. Baghabra Al-Amoudi, A review on the angle of repose of granular materials, Powder Technol. 330 (2018) 397-417. https://doi.org/10.1016/j.powtec.2018.02.003.
[36] Y. Zhang, M. Huo, J. Zhou, S. Xie, PKSolver: An add-in program for pharmacokinetic and pharmacodynamic data analysis in Microsoft Excel, Comput. Methods Programs Biomed. 99 (2010) 306-314. https://doi.org/10.1016/j.cmpb.2010.01.007.

1. A fully dilutable in aqueous phase self-microemulsifying system for the delivery of one or more polar oil active compounds having a positive characteristic curvature (Cc), comprising:
a lecithin compound;
a hydrophilic linker (HL) or a combination of two or more HLs, the HL or each of the HLs within the combination having one hydrocarbon group with at least 50% or more alkyl chain distribution between 6 to 10 carbon atoms, and the HL or the combination of two or more HLs having a Cc of about −5 or more negative than about −5; and
a carrier oil,
wherein the system is free of polyethylene glycol.

2. The fully dilutable in aqueous phase self-microemulsifying composition of claim 1, wherein the delivery is topical, transdermal, oral, transnasal, buccal, vaginal, subcutaneous, parenteral, ophthalmic, transepidermal, transmembrane, and intravenous.

3. The fully dilutable in aqueous phase self-microemulsifying system of claim 1, wherein the lecithin compound concentration is about 1.5% to about 45% w/w.

4. (canceled)

5. The fully dilutable in aqueous phase self-microemulsifying system of claim 1, wherein the hydrophilic linker is about 10 wt % to about 86 wt %.

6. The fully dilutable in aqueous phase self-microemulsifying system of claim 1, wherein the combination of two or more HLs includes at least one amphiphilic compound with a Cc less negative than about −5 and the Cc of the combination is about −5 or more negative than about −5.

7. The fully dilutable in aqueous phase self-microemulsifying system of claim 1, wherein the hydrophilic linker or the combination of two or more HLs comprises one or more of C6-C10 alkyl polyphosphates, polyphosphonates, polycarboxylates, sulfosuccinates, glutamates; C6-C10 esters of polyhydric alcohols, polyvinyl alcohol, polyglycerols and their co-polymers with a degree of polymerization (n) higher than 2 (n>2), sucrose, maltose, oligosaccharides, polyglucosides (n>2), polyglucosamines, sorbitol, sorbitan, poly alpha hydroxy acids and their salts, C6-C10 amines, quaternary ammonium salts, amine oxides, C6-C10 alkyl aminopropionic acids, betaines, sulfobetaines, phosphatidylcholines, phosphatidyl glycerols, or mixtures thereof.

8. The fully dilutable in aqueous phase self-microemulsifying system of claim 1, wherein the hydrophilic linker or at least one of the two or more HLs in the combination comprises a C6-C10 polyglycerol with a degree of polymerization n>2, or wherein the hydrophilic linker or at least one of the two or more HLs in the combination is disodium C6-C10 glutamate, polyglycerol-6-caprylate or polyglycerol-10 caprylate.

9. (canceled)

10. The fully dilutable in aqueous phase self-microemulsifying system of claim 1, wherein the carrier oil has a positive equivalent alkane carbon number (EACN).

11. The fully dilutable in aqueous phase self-microemulsifying system of claim 1, wherein the carrier oil concentration is about 10 wt % to about 70 wt %.

12. The fully dilutable in aqueous phase self-microemulsifying system of claim 1, wherein the carrier oil comprises of alkyl esters of fatty acids, monoglycerides, diglycerides, triglycerides, alkanes, terpenes, or mixtures thereof.

13. The fully dilutable in aqueous phase self-microemulsifying system of claim 1, wherein the self-microemulsifying system further includes the one or more polar oil active compounds having a positive characteristic curvature (Cc).

14. The fully dilutable in aqueous phase self-micoremulsifying system of claim 13, wherein the concentration of the one or more polar oil active compounds is about 0.01 wt % to about 80 wt %.

15. The fully dilutable in aqueous phase self-microemulsifying system of claim 13, wherein each of the one or more polar oil active compounds having a positive characteristic curvature (Cc) has a log P greater than 1, molecular weight between 50 and 100,000 Daltons, a polar area greater than 0.0 Å², an aqueous solubility less than about 1 wt %.

16. The fully dilutable in aqueous phase self-microemulsifying system of claim 13, wherein the one or more polar oil active compounds having a positive characteristic curvature (Cc) includes one or more hydrogen bonding donor compounds selected from a group consisting of C5+ alcohols, amines, peptides, organic acids, anthranilic acids, aryl propionic acids, enolic acids, heteroaryl acetic acids, indole and indene acetic acids, salicylic acid derivatives, nucleic acids, alkylphenols, para-aminophenol derivatives, terpene phenolics, cannabinoids, alkaloids, peptides, and halogenated compounds.

17. The fully dilutable in aqueous phase self-microemulsifying system of claim 13, wherein the one or more polar active compounds include ibuprofen, nonylphenol, cannabidiol, and eugenol.

18. The fully dilutable in aqueous phase self-microemulsifying system of claim 1, wherein the aqueous phase is water, biological fluids, aqueous electrolyte solutions, carbonated drinks, fruit juices, or alcoholic beverages.

19. The fully dilutable in aqueous phase self-microemulsifying system of claim 1, wherein the system further comprises a lipophilic linker.

20. The fully dilutable in aqueous phase self-microemulsifying system of claim 19, wherein the lipophilic linker concentration is about 0.1 wt % to about 30.0 wt %.

21. The fully dilutable in aqueous phase self-microemulsifying system of claim 19, wherein the lipophilic linker includes one or more ingredients selected from a group consisting of C12+ alcohols, fatty acids, monoglyceride, sorbitan ester, sucrose ester, glucose ester, cholesterol, and esters of lauric acid, palmitic acid, oleic acid, omega 6-fatty acids, omega 3-fatty acids with sorbitol, maltitol, xylitol, isomalt, lactitol, erythritol, pentaerythritol, and glycerol.

22. (canceled)

23. The fully dilutable in aqueous phase self-microemulsifying system of claim 1, wherein the system further comprises a low molecular weight organogelator that imparts semisolid properties and produces a slow releasing profile of the one or more polar oil active compounds, wherein the concentration of the organogelator is about 0.1 wt % to about 40.0 wt %, and wherein the organogelator includes one or more ingredients selected from sterol-based gelling agents, long-chain fatty acids, long-chain amines, and esters of long-chain fatty acids.

24-25. (canceled)

26. The fully dilutable in aqueous phase self-microemulsifying system of claim 1, wherein the system further comprises an encapsulating agent that imparts solid-like properties and produce flowable powders that can form micellar solutions when diluted in aqueous environments.

27. The fully dilutable in aqueous phase self-microemulsifying system of claim 26, wherein the concentration of the encapsulating agent is about 10% to about 90.0% wt.

28. The fully dilutable in aqueous phase self-microemulsifying system of claim 26, wherein the encapsulating agent is a polymer with a glass transition temperature ranging from about 45° C. to about 99° C.

29-30. (canceled)

31. The fully dilutable in aqueous phase self-microemulsifying system of claim 1, wherein the system is waterless, and wherein the system has particle diameters smaller than 200 nm.

32-35. (canceled)

36. A method of delivering one or more polar oil active compounds having a positive characteristic curvature (Cc) to a subject comprising administering to a subject the fully dilutable in aqueous phase self-microemulsifying system of claim 1.

37-67. (canceled)

68. The fully dilutable in aqueous phase self-microemulsifying composition of claim 1,
wherein the fully dilutable in aqueous phase self-microemulsifying composition is further free of propylene glycol, and short and medium-chain alcohols.
